(19) United States
(12) Patent Application Publication
(10) Pub. No.: US 2005/0182011 A1
(43) Pub. Date: Aug. 18, 2005

(54) Inhibition of TRP channels as a treatment for cardiac hypertrophy and heart failure

(76) Inventors: Eric Olson, Dallas, TX (US); Erik Bush, Westminster, CO (US)

Correspondence Address: FULBRIGHT & JAWORSKI L.L.P., 600 CONGRESS AVE., SUITE 2400, AUSTIN, TX, 78701, US

(21) Appl. No.: 10/988,192

(22) Filed: Nov. 12, 2004

Related U.S. Application Data
(60) Provisional application No. 60/519,980, filed on Nov. 13, 2003.

(51) Int. Cl. A61K 48/00; A61K 38/16; A61K 39/395; A61K 31/555

(52) U.S. Cl. 514/44; 424/143.1; 514/2; 514/6; 514/184; 514/102

(57) ABSTRACT

The present invention provides methods of treating and preventing cardiac hypertrophy and heart failure. MEF-2, NF-AT3, calcineurin, MCIP, and Class II HDACs have been shown to have a major role in cardiac hypertrophy and heart disease, and inhibition of many of these factors or the pathways mediated by these factors has been shown to have a beneficial, anti-hypertrophic effect. The present invention provides a link between these factors and the pathways they mediate through a family of non-voltage gated channels called TRP channels. The present invention further demonstrates that inhibitors of TRP channels can inhibit or treat heart failure and cardiac hypertrophy.

This application claims priority to U.S. Provisional Patent Application 60/519,980 filed on Nov. 13, 2003, which is specifically incorporated by reference.

### BACKGROUND OF THE INVENTION

1. Field of the Invention

The present invention relates generally to the fields of developmental biology and molecular biology. More particularly, it concerns gene regulation and cellular physiology in cardiomyocytes. Specifically, the invention relates to the use inhibitors of Transient Receptor Potential (TRP) channels to block non-voltage gated calcium flux into cells. It also relates to the use of TRP channel inhibitors to treat cardiac hypertrophy and heart failure, and to screening methods for finding inhibitors of cardiac TRP channels.

2. Description of Related Art

Cardiac hypertrophy is an adaptive response of the heart to many forms of cardiac disease, including hypertension, mechanical load abnormalities, myocardial infarction, valvular dysfunction, certain cardiac arrhythmias, endocrine disorders and genetic mutations in cardiac contractile protein genes. While the hypertrophic response is thought to be an initially compensatory mechanism that augments cardiac performance, sustained hypertrophy is maladaptive and frequently leads to ventricular dilation and the clinical syndrome of heart failure. Accordingly, cardiac hypertrophy has been established as an independent risk factor for cardiac morbidity and mortality (Levy et al., 1990).

Diverse hypertrophic stimuli such as pressure overload or adrenergic agonists induce a stereotypical pattern of changes in cardiac gene expression that include the re-expression of fetal genes such as atrial natriuretic factor, alpha skeletal actin and beta myosin heavy chain (Chein et al., 1993; Sadoshima et al., 1997). Regardless of the stimulus, increased concentrations of intracellular calcium appear to function as a common proximal signal for the initiation of hypertrophic gene expression. (Olson and Williams, 2000a; Olson and Williams, 2000b). One major downstream effector of this signal is the calcium-dependent phosphatase calcineurin, which plays a critical role in the promotion of cardiac hypertrophy. Activated calcineurin dephosphorylates the transcription factor NFAT, which then enters the nucleus and promotes hypertrophic gene expression (Molkenti et al., 1998). This core signaling module (calcium to calcineurin to NFAT) functions in a variety of vertebrate cell types (Crabtree and Olson, 2002).

The intracellular compartment normally maintains low concentrations (100 nM) of calcium relative to the extracellular environment (1 mM) or internal (sarcoplasmic reticulum) stores. Transient increases in intracellular calcium concentrations (such as those associated with the cardiac excitation-contraction cycle) are insufficient to activate calcineurin; rather, calcineurin responds to persistent elevations in intracellular calcium. While hypertrophic cardiomyocytes clearly possess chronically elevated intracellular calcium levels, the specific mechanisms responsible for this persistent calcium signal remain elusive. Potential mechanisms may include increased extracellular calcium entry, increased calcium release from internal stores or impaired reuptake of calcium via the SERCA pump. Extracellular calcium entry is regulated primarily by cardiac L-type voltage-gated channels, and to a lesser degree, by a variety of non-voltage-gated calcium channels. The ryanodine receptor mediates the majority of calcium released from the sarcoplasmic reticulum during the exitation-contraction cycle, and is 50- to 100-fold more abundant in the heart than another calcium release channel, the IP3 receptor. Despite its lower abundance, recent evidence suggests that the IP3 receptor may play a key role in promoting the cardiac calcineurin-NFAT pathway (Jayaraman and Marks, 2000). Furthermore, increases in IP3 receptor expression have been observed in human patients with heart failure (Go et al., 1995).

Additional insights into the possible origin of the hypertrophic calcium signal have come from studies of the calcineurin-NFAT pathway in the immune system (Crabtree and Olson, 2002). During lymphocyte activation, ligand binding to T-cell receptors stimulates PLC activation and the production of IP3, which induces a transient release of calcium from intracellular stores via the IP3 receptor (the predominant calcium release channel in lymphocytes). This transient calcium release, however, is insufficient to activate calcineurin and subsequent NFAT-dependent responses. Rather, the initial calcium release from intracellular stores triggers a secondary influx of extracellular calcium through specialized Calcium Release Activated Calcium (CRAC) channels. It is this influx of extracellular calcium that produces the sustained calcium signal capable of activating the calcineurin pathway. Given the degree to which the calcineurin-NFAT signaling module is utilized in a variety of cell types, it is reasonable to predict that a similar mechanism (e.g., a cardiac CRAC channel) may be responsible for activation of this pro-hypertrophic pathway in the heart.

While the electrophysiologic characteristics of cardiac CRAC channels have been extensively studied, the specific genes encoding these channels have yet to be completely identified. Thus, although the gene or genes responsible for cardiac CRAC channel characteristics represent a starting point for the cascade leading to hypertrophy and are potential therapeutic targets for both heart failure and hypertrophy, their genetic identity remains obscure.

### SUMMARY OF THE INVENTION

Thus, in accordance with the present invention, there is provided a method of treating cardiac hypertrophy or heart failure comprising (a) identifying a patient having cardiac hypertrophy or heart failure; and (b) administering to the patient an inhibitor of a TRP channel. In various embodiments, the TRP channel may be a TRPC family channel, and in further embodiments it may be a TRPC1, TRPC3, TRPC4, TRPC5 or TRPC6 channel.

In certain embodiments of the invention, the inhibitor may be selected from the group consisting of an antibody, an RNAi molecule, a ribozyme, a peptide, a small molecule, an antisense molecule, 2-ABP, D-myo-1-INS(1,4,5)P₃, gadolinium, Anti-G(q/11) antibody, U-73122, La³⁺, flufanemate, PPI, lanthanum, or condensed cortical F-actin. In further embodiments, the antibody selected may be monoclonoal, polyclonal, humanized, single chain or an Fab fragment.

Administering may comprise intravenous, oral, transdermal, sustained release, suppository, or sublingual administration. The method may further comprise administering a second therapeutic regimen, such as a beta blocker, an iontrope, diuretic, ACE-I, AII antagonist, a histone deacetylase inhibitor, or Ca(++)-blocker. The second therapeutic regimen may be administered at the same time as the inhibitor, or either before or after the inhibitor.

The treatment may improve one or more symptoms of cardiac hypertrophy or heart failure, such as providing increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, wall tension and wall thickness, quality of life, disease-related morbidity and mortality, reversal of progressive remodeling, improvement of ventricular dilation, increased cardiac output, relief of impaired pump performance, improvement in arrhythmia, fibrosis, necrosis, energy starvation or apoptosis.

In another embodiment of the invention, there is provided a method of preventing cardiac hypertrophy or heart failure comprising (a) identifying a patient at risk for cardiac hypertrophy or heart failure; and (b) administering to said patient an inhibitor of a TRP channel. The TRP channel may be a TRPC channel, and more particularly it will be a TRPC1, TRPC3, TRPC4, TRPC5 or TRPC6 channel.

Administration may comprise intravenous, oral, transdermal, sustained release, suppository, or sublingual administration. The patient may exhibit one or more of long standing uncontrolled hypertension, uncorrected valvular disease, chronic angina, or have experienced a recent myocardial infarction.

In certain embodiments of the invention the inhibitor may be selected from the group consisting of an antibody, an RNAi molecule, a ribozyme, a peptide, a small molecule, an antisense molecule, 2-ABP, D-myo-1-INS(1,4,5)P₃, gadolinium, Anti-G(q/11) antibody, U-73122, La³⁺, flufanemate, PPI, lanthanum, or condensed cortical F-actin. In further embodiments, the antibody selected may be monoclonoal, polyclonal, humanized, single chain or an Fab fragment.

In yet another embodiment of the invention, there is provided a method for identifying an inhibitor of a TRPC channel in a cardiac cell comprising (a) providing a cardiomyocyte; (b) contacting said cardiomyocyte with a candidate inhibitor substance; and (c) measing an activity mediated by a TRPC channel on said cardiomyocyte; wherein a decrease in cardiomyocyte TRPC channel activity, as compared to TRPC channel activity measured in an untreated cell, identifies the candidate substance as an inhibitor of cardiac TRPC channel activity. In particular embodiments of the invention, the activity mediated by a TRPC channel that is measured comprises non-voltage gated calcium flux, calcineurin enzymatic activity, MCIP protein levels, MCIP RNA levels, or NF-AT3-mediated gene expression.

In certain embodiments of the invention, the TRPC channels will be located in intact cells, either endogenously or by induced over-expression. The cardiomyocytes may be neonatal rat ventricular myocytes. The cardiomyocytes may further be located in an intact heart, and that heart may be a human heart.

In yet another embodiment of the invention, there is provided a method for indentifying an inhibitor of heart failure or hypertrophy comprising (a) providing a TRP channel inhibitor; (b) treating a myocyte with that TRP channel inhibitor; and (c) measuring the expression of one or more cardiac hypertrophy or heart failure parameters, wherein a change in said one or more cardiac hypertrophy or heart failure parameters, as compared to one or more cardiac hypertrophy or heart failure parameters in an untreated myocyte, identifies said TRP channel inhibitor as an inhibitor of heart failure or cardiac hypertrophy. Further, the myocyte may be subjected to a stimulus that triggers a hypertrophic response in the one or more cardiac hypertrophy parameters, such as expression of a transgene or treatment with a chemical agent.

The one or more cardiac hypertrophy parameters may comprise the expression level of one or more target genes in the myocyte, wherein the expression level of the one or more target genes is indicative of cardiac hypertrophy. The one or more target genes may be selected from the group consisting of ANF, α-MyHC, β-MyHC, α-skeletal actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen. The expression level may be measured using a reporter protein coding region operably linked to a target gene promoter, such as luciferase, β-galactosidase or green fluorescent protein. The expression level may be measured using hybridization of a nucleic acid probe to a target mRNA or amplified nucleic acid product.

The one or more cardiac hypertrophy parameters also may comprise one or more aspects of cellular morphology, such as sarcomere assembly, cell size, or cell contractility. The myocyte may be an isolated myocyte, or comprised in isolated intact tissue. The myocyte also may be a cardiomyocyte, and may be located in vivo in a functioning intact heart muscle, such as functioning intact heart muscle that is subjected to a stimulus that triggers heart failure or a hypertrophic response in one or more cardiac hypertrophy parameters. The cardiomyocyte may be a neonatal rat ventricular myocyte (NRVM). The stimulus may be aortic banding, rapid cardiac pacing, induced myocardial infarction, osmotic minipumps, PTU treatment, induced diabetes, or transgene expression. The one or more cardiac hypertrophy parameters comprises right ventricle ejection fraction, left ventricle ejection fraction, ventricular wall thickness, heart weight/body weight ratio, or cardiac weight normalization measurement. The one or more cardiac hypertrophy parameters also may comprise total protein synthesis.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Diverse hypertrophic stimuli increase TRPC3 protein expression in cultured cardiomyocytes. Western blot analysis with anti-TRPC3 primary on protein isolated from unstimulated NRVM and NRVM stimulated with phenylephrine (20 mM), fetal bovine serum (10%) or adenovirus encoding constitutively active calcineurin (multiplicity of infection=25).

FIGS. 2A-B—Cardiac TRPC3 protein expression is increased in an in vivo model of pressure-overload hypertrophy. (FIG. 2A) Western blot analysis with anti-TRPC3 primary on left ventricular protein isolated from sham-operated animals and animals subjected to thoracic aortic banding. Loading equivalency verified by sequential Western blot with a primary antibody to the IP90 housekeeping gene. (FIG. 2B) Quantitation of TRPC3 signal by densitometry.

FIGS. 3A-B—Cardiac TRPC3 expression is increased in vivo in a pharmacologic model of hypertrophy. (FIG. 3A) Western blot analysis with anti-TRPC3 primary on left ventricular protein isolated from animals chronically infused with saline (control) or isoproterenol. Loading equivalency verified by sequential Western blot with a primary antibody to the IP90 housekeeping gene. (FIG. 3B) Quantitation of TRPC3 signal by densitometry.

FIGS. 4A-B—Cardiac TRPC3 and TRPC1 protein expression is increased in a genetic model of hypertrophy and heart failure. (FIG. 4A) Western blot analysis with anti-TRPC3 and anti-TRPC1 primary antibodies on left ventricular protein isolated from 2 month-old, 8-9 month-old and 19 month-old SHHF rats. (FIG. 4B) Quantitation of TRPC3 and TRPC1 signals by densitometry.

FIG. 5—Compound 2-APB produces no significant cytotoxicity in cultured cardiomyocytes. Quantitation of cytotoxicity by adenylate kinase (AK) release in NRVM cultured with increasing concentrations of 2-APB for a period of 48 hours. Positive control for cytotoxicity provided by treating NRVM with 0.1% Triton X-100 (dotted line, approximately 6-fold increase). Data plotted as-fold change in AK release versus unstimulated, no 2-APB control (±S.E.).

FIG. 6—Compound 2-APB attenuates PE-dependent induction of ANF secretion. Quantitation of ANF secretion in unstimulated and PE-stimulated NRVM exposed to increasing concentrations of 2-APB for a period of 48 hours. Data plotted as ng/ml ANF (±S.E.).

FIG. 7—Compound 2-APB attenuates PE-dependent induction of calcineurin-regulated 28 kDa MCIP1 protein. Western blot analysis with anti-MCIP1 primary on protein isolated from unstimulated NRVM (left panel) and PE-stimulated NRVM (right panel) in the presence of increasing concentrations of 2-APB.

FIG. 8—Compound 2-APB attenuates PE-dependent increases in total cellular protein. Quantitation of total cellular protein in unstimulated NRVM and PE-stimulated NRVM exposed to increasing concentrations of 2-APB for a period of 48 hours. Data plotted as total protein absorbance at A595 (±S.E.).

FIG. 9—Compound 2-APB attenuates PE-dependent increases in cardiomyocyte volume. Cell volume measurements of unstimulated NRVM and PE-stimulated NRVM exposed to increasing concentrations of 2-APB for a period of 48 hours. Data plotted as cell volume in femtoliters (±S.E.).

FIG. 10A-B—Cardiac TRPC5 expression is increased in the failing human heart. FIG. 10A—Western blot analysis with anti-TRPC5 primary on left ventricular protein isolated from non-failing and failing human hearts. Loading equivalency verified by sequential Western blot with a primary antibody to the IP90/calnexin housekeeping gene. FIG. 10B—Quantitation of TRPC5 signal by densitometry.

### DESCRIPTION OF ILLUSTRATIVE EMBODIMENTS

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce heart failure, the symptoms of cardiomyopathy are those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

In light of the limitations of the current therapies, the inventors discovered a novel set of proteins that are substantially upregulated in failing hearts, hypertrophic hearts and hypertrophic tissues. Using a genechip anaylsis, the inventors identified TRPC3 and TRPC1 as genes that were upregulated in response to prohypertophic stimuli. Analysis of failing heart tissue, and further experiments in vitro described herein, have shown the TRP family channels are an excellent therapeutic target. These non-voltage gated Ca(++) channels are the starting point for a number of important signaling pathways already known to be important in the cellular cascade towards hypertrophy. Thus, in accordance with the present invention, the inventors describe herein novel therapeutic methods for treating cardiac hypertrophy and heart failure by inhibiting TRP channel function.

I. TRP Channels

As previously stated, while the electrophysiologic characteristics of CRAC channels have been extensively studied, the specific genes encoding these channels have yet to be identified. However, the channel protein CaT1 has recently been demonstrated to possess the expected electrophysiologic properties of a CRAC channel (Yue et al., 2001). CaT1 is a member of a large group (approximately 20 genes) of non-voltage-gated plasma membrane cation channels collectively known as the Transient Receptor Potential (TRP) family (Venneken et al., 2002). The TRP family can be divided into three subfamilies on the basis of sequence homology: the TRPC (canonical) subfamily, the TRPV (vanilloid) subfamily and the TRPM (melastatin) subfamily. TRP family members clearly function as calcium influx channels in a variety of tissues, but relatively little is currently known about the specific physiological roles and modes of regulation of this emerging ion channel family.

Members of the TRPC subfamily are known effectors of G-protein coupled receptors, and are directly activated by diacylglycerol and IP3 produced as a result of GPCR-dependent PLC activation. TRPC subfamily members also function as CRAC channels; they are activated in response to depletion of intracellular calcium stores. The specific mechanism coupling store depletion to calcium influx is unknown, but in the case of TRPC3, the channel is thought to interact directly with the IP3 receptor. Interestingly, expression level of the TRPC3 channel has been shown to influence how the channel is regulated; PLC activation is the predominant regulatory mode at high levels of channel expression, while lower expression levels favor store depletion (Vasquez et al., 2003). Crucially, TRPC channels have recently been demonstrated to contribute to pathologic calcium signaling in muscle (Vandebrouck et al., 2002). Skeletal muscle fibers from patients suffering from Duchenne muscular dystrophy exhibit abnormally increased calcium influx, which contributes to the dystrophic phenotype via activation of calcium-dependent proteases. Antisense repression of TRPC expression in dystrophic muscle fibers reduced the abnormal calcium influx, confirming the role of this channel in the disease process.

Other TRP subfamily members are less well studied, but appear to respond to different stimuli. In addition to regulation by store depletion, TRPV channels are also activated by mechanical stretch, heat and the hot pepper compound capsaicin. In contrast, TRPM channels are activated by cold temperatures and compounds like menthol. Although expressed in muscle, the functional roles these channels may play have yet to be described. Table 1 provides a list of accession numbers for known TRP channels.

TABLE 1

| Human channel isoform | mRNA accession # | protein accession # |
|---|---|---|
| TRPC1 | NM_003304 | NP_003295 |
| TRPC3 | NM_003305 | NP_003296 |
| TRPC4 | NM_016179 | NP_057263 |
| TRPC5 | NM_012471 | NP_036603 |
| TRPC6 | NM_004621 | NP_004612 |
| TRPC7 | NM_020389 | NP_065122 |
| TRPV1 | NM_080704 | NP_542435 |
| TRPV2 | NM_016113 | NP_057197 |
| TRPV4 | NM_021625 | NP_067638 |
| TRPV5 | NM_019841 | NP_062815 |
| TRPV6 | NM_018646 | NP_061116 |
| TRPM2 | NM_003307 | NP_003298 |
| TRPM3 | NM_020952 | NP_066003 |
| TRPM4 | NM_017636 | NP_060106 |
| TRPM5 | NM_014555 | NP_055370 |
| TRPM6 | NM_017662 | NP_060132 |
| TRPM7 | NM_017672 | NP_060142 |
| TRPM8 | NM_024080 | NP_076985 |

II. Heart Failure and Hypertrophy

Heart disease and its manifestations, including coronary artery disease, myocardial infarction, congestive heart failure and cardiac hypertrophy, clearly presents a major health risk in the United States today. The cost to diagnose, treat and support patients suffering from these diseases is well into the billions of dollars. One particularly severe manifestations of heart disease is cardiac hypertrophy. Regarding hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been fully elucidated. Understanding these mechanisms is a major concern in the prevention and treatment of cardiac disease and will be crucial as a therapeutic modality in designing new drugs that specifically target cardiac hypertrophy and cardiac heart failure. The symptoms of cardiac hypertrophy initially mimic those of heart failure and may include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of hypertrophy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

MEF-2, MCIP, Calcineurin, NF-AT3, and Histone Deactylases (HDACs) are all proteins and genes that have been recently implicated as intimately involved in the development of and progression of heart disease, heart failure, and hypertrophy. Manipulation, modulation, and/or inhibition of any or all of these genes and/or proteins holds great promise in the treatment of heart failure and hypetrophy. These genes are all involved in a variety of cascades that eventually lead to both heart failure and hypertrophy. As such, if there was a way to inhibit these genes at the top of the cascade, to perhaps prevent the activation of these genes in the first place, that would represent a significant leap in the treatment of cardiac disease. The TRP channels are such a potential target, for they are associated with all of these cascades as a starting point, a therapeutic bottleneck, for inhibiting the transcriptional and translational pathways associated with heart failure and hypertrophy.

III. Transcriptional Pathway for Heart Failure or Cardiac Hypertrophy

It is known that Ca(++) activation is involved in a variety of forms of heart failure and heart disease. Ca(++) store depletion, or a raise in the cytoplasmic Ca(++) levels in the cell, has been show to stimulate a calcineurin dependent pathway for cardiac hypertrophy. The inventors show that TRP channels are the putative channels responsible for raising these intracellular Ca(++) levels, which then activates a number of different pathways in the cell. The individual components of these pathways as they relate to cardiac hypertrophy are discussed in further detail herein below.

A. Calcineurin

Calcineurin is a ubiquitously expressed serine/threonine phosphatase that exists as a heterodimer, comprised of a 59 kD calmodulin-binding catalytic A subunit and a 19 kD Ca(++)-binding regulatory B subunit (Stemmer and Klee, 1994; Su et al., 1995). Calcineurin is uniquely suited to mediate the prolonged hypertrophic response of a cardiomyocyte to Ca(++) signaling because the enzyme is activated by a sustained Ca(++) plateau and is insensitive to transient Ca(++) fluxes as occur in response to cardiomyocytc contraction (Dolmetsch et al., 1997).

Activation of calcineurin is mediated by binding of Ca(++) and calmodulin to the regulatory and catalytic subunits, respectively. Previous studies showed that over-expression of calmodulin in the heart also results in hypertrophy, but the mechanism involved was not determined (Gruver et al., 1993). It is now clear that calmodulin acts through the calcineurin pathway to induce the hypertrophic response. Calcineurin has been shown previously by the inventors to phosphorylate NF-AT3, which subsequently acts on the transcription factor MEF-2 (Olson and Williams, 2000). Once this event occurs, MEF-2 activates a variety of genes known as fetal genes, the activation of which inevitably results in hypertrophy.

CsA and FK-506, bind the immunophilins cyclophilin and FK-506-binding protein (FKBP12), respectively, forming complexes that bind the calcineurin catalytic subunit and inhibit its activity. CsA and FK-506 block the ability of cultured cardiomyocytes to undergo hypertrophy in response to AngII and PE. Both of these hypertrophic agonists have been shown to act by elevating intracellular Ca(++), which results in activation of the PKC and MAP kinase signaling pathways (Sadoshima et al., 1993; Sadoshima and Izumo, 1993; Kudoh et al., 1997; Yamazaki et al., 1997, Zou et al., 1996). CsA does not interfere with early signaling events at the cell membrane, such as PI turnover, Ca(++) mobilization, or PKC activation (Emmel et al., 1989). Thus, its ability to abrogate the hypertrophic responses of AngII and PE suggests that calcineurin activation is an essential step in the AngII and PE signal transduction pathways.

B. NF-AT3

NF-AT3 is a member of a multigene family containing four members, NF-ATc, NF-ATp, NF-AT3, and NF-AT4 (McCaffery et al., 1993; Northrup et al., 1994; Hoey et al., 1995; Masuda et al., 1995; Park et al., 1996; Ho et al., 1995). These factors bind the consensus DNA sequence GGAAAAT as monomers or dimers through a Rel homology domain (RHD) (Rooney et al., 1994; Hoey et al., 1995). Three of the NF-AT genes are restricted in their expression to T-cells and skeletal muscle, whereas NF-AT3 is expressed in a variety of tissues including the heart (Hoey et al., 1995). For additional disclosure regarding NF-AT proteins the skilled artisan is referred to U.S. Pat. No. 5,708,158, specifically incorporated herein by reference.

NF-AT3 is a 902-amino acid with a regulatory domain at its amino-terminus that mediates nuclear translocation and the Rel-homology domain near its carboxyl-terminus that mediates DNA binding. There are three different steps involved in the activation of NF-AT proteins, namely, dephosphorylation, nuclear localization and an increase in affinity for DNA. In resting cells, NFAT proteins are phosphorylated and reside in the cytoplasm. These cytoplasmic NF-AT proteins show little or no DNA affinity. Stimuli that elicit calcium mobilization result in the rapid dephosphorylation of the NF-AT proteins and their translocation to the nucleus. The dephosphorylated NF-AT proteins show an increased affinity for DNA. Each step of the activation pathway may be blocked by CsA or FK506. This implies, and the inventors earlier studies have shown, that calcineurin is the protein responsible for NF-AT activation.

Thus, in T cells, many of the changes in gene expression in response to calcineurin activation are mediated by members of the NF-AT family of transcription factors, which translocate to the nucleus following dephosphorylation by calcineurin. Many observations support the conclusion that NF-AT also is an important mediator of cardiac hypertrophy in response to calcineurin activation. NF-AT activity is induced by treatment of cardiomyocytes with AngII and PE. This induction is blocked by CsA and FK-506, indicating that it is calcineurin-dependent. NF-AT3 synergizes with GATA4 to activate the cardiac specific BNP promoter in cardiomyocytes. Also, expression of activated NF-AT3 in the heart is sufficient to bypass all upstream elements in the hypertrophic signaling pathway and evoke a hypertrophic response.

The inventors' prior work demonstrates that the C-terminal portion of the Rel-homology domain of NF-AT3 interacts with the second zinc finger of GATA4, as well as with GATA5 and GATA6, which are also expressed in the heart. The crystal structure of the DNA binding region of NF-ATc has revealed that the C-terminal portion of the Rel-homology domain projects away from the DNA binding site and also mediates interaction with AP-1 in immune cells (Wolfe et al., 1997).

According to a model previously proposed by the inventors, hypertrophic stimuli such as AngII and PE, which lead to an elevation of intracellular Ca(++), result in activation of calcineurin. NF-AT3 within the cytoplasm is dephosphorylated by calcineurin, enabling it to translocate to the nucleus where it can interact with GATA4, and then activate the transcription factor MEF-2, a family of transcription factors that are normally repressed by a tight association with class II HDAC's.

Results of previous work by the inventors has shown that calcineurin activation of NF-AT3 regulates hypertrophy in response to a variety of pathologic stimuli and suggests a sensing mechanism for altered sarcomeric function. Of note, there are several familial hypertrophic cardiomyopathies (FHC) caused by mutations in contractile protein genes, which result in subtle disorganization in the fine crystalline-like structure of the sarcomere (Watkins et al., 1995; Vikstrom and Leinwand, 1996). It is unknown how sarcomeric disorganization is sensed by the cardiomyocyte, but it is apparent that this leads to altered Ca(++) handling (Palmiter and Solaro, 1997; Botinelli et al., 1997; Lin et al., 1996). Calcineurin, as discussed above, is one of the sensing molecules that couples altered Ca(++) handling associated with FHC with cardiac hypertrophy and heart failure.

C. MEF2

As mentioned above, NF-AT3 activation by Calcineurin leads to the activation of another family of transcription factors, the monocyte enhancer factor-2 family (MEF2), which are known to play an important role in morphogenesis and myogenesis of skeletal, cardiac, and smooth muscle cells (Olson et al., 1995). MEF2 factors are expressed in all developing muscle cell types, binding a conserved DNA sequence in the control regions of the majority of muscle-specific genes. Of the four mammalian MEF2 genes, three (MEF2A, MEF2B and MEF2C) can be alternatively spliced, which have significant functional differences (Brand, 1997; Olson et al., 1995). These transcription factors share homology in an N-terminal MADS-box and an adjacent motif known as the MEF2 domain. Together, these regions of MEF2 mediate DNA binding, homo- and heterodimerization, and interaction with various cofactors, such as the myogenic bHLH proteins in skeletal muscle. Additionally, biochemical and genetic studies in vertebrate and invertebrate organisms have demonstrated that MEF2 factors regulate myogenesis through combinatorial interactions with other transcription factors.

Loss-of-function studies indicate that MEF2 factors are essential for activation of muscle gene expression during embryogenesis. The expression and functions of MEF2 proteins are subject to multiple forms of positive and negative regulation, serving to fine-tune the diverse transcriptional circuits in which the MEF2 factors participate. MEF-2 is bound in an inactive form in the healthy heart by class II HDACS (see supra), and when MEF-2 is activated it is released from the HDAC and activates the fetal gene program that is so deleterious for the heart.

D. Histone Deacetylase

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

Eleven different HDACs have been cloned from vertebrate organisms. The first three human HDACs identified were HDAC 1, HDAC 2 and HDAC 3 (termed class I human HDACs), and HDAC 8 (Van den Wyngaert et al., 2000) has been added to this list. Recently class II human HDACs, HDAC 4, HDAC 5, HDAC 6, HDAC 7, HDAC 9, and HDAC 10 (Kao et al., 2000) have been cloned and identified (Grozinger et al., 1999; Zhou et al. 2001; Tong et al., 2002). Additionally, HDAC 11 has been identified but not yet classified as either class I or class II (Gao et al., 2002). All share homology in the catalytic region. HDACs 4, 5, 7, 9 and 10 however, have a unique amino-terminal extension not found in other HDACs. This amino-terminal region contains the MEF2-binding domain. HDACs 4, 5 and 7 have been shown to be involved in the regulation of cardiac gene expression and in particular embodiments, repressing MEF2 transcriptional activity. The exact mechanism in which class II HDAC's repress MEF2 activity is not completely understood. One possibility is that HDAC binding to MEF2 inhibits MEF2 transcriptional activity, either competitively or by destabilizing the native, transcriptionally active MEF2 conformation. It also is possible that class II HDAC's require dimerization with MEF2 to localize or position HDAC in a proximity to histones for deacetylation to proceed.

A variety of inhibitors for histone deacetylase have been identified. The proposed uses range widely, but primarily focus on cancer therapy (Saunders et al., 1999; Jung et al., 1997; Jung et al., 1999; Vigushin et al., 1999; Kim et al., 1999; Kitazomo et al., 2001; Vigusin et al., 2001; Hoffmann et al., 2001; Kramer et al., 2001; Massa et al., 2001; Komatsu et al., 2001; Han et al., 2001). Such therapy is the subject of NIH sponsored clinical trials for solid and hematological tumors. HDAC's also increase transcription of transgenes, thus constituting a possible adjunct to gene therapy. (Yamano et al., 2000; Su et al., 2000).

HDACs can be inhibited through a variety of different mechanisms—proteins, peptides, and nucleic acids (including antisense, RNAi molecules, and ribozymes). Methods are widely known to those of skill in the art for the cloning, transfer and expression of genetic constructs, which include viral and non-viral vectors, and liposomes. Viral vectors include adenovirus, adeno-associated virus, retrovirus, vaccina virus and herpesvirus.

Also contemplated are small molecule inhibitors. Perhaps the most widely known small molecule inhibitor of HDAC function is Trichostatin A, a hydroxamic acid. It has been shown to induce hyperacetylation and cause reversion of ras transformed cells to normal morphology (Taunton et al., 1996) and induces immunsuppression in a mouse model (Takahashi et al., 1996). It is commercially available from a variety of sources including BIOMOL Research Labs, Inc., Plymouth Meeting, Pa.

The following references, incorporated herein by reference, all describe HDAC inhibitors that may find use in the present invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. 2002/256221; U.S. 2002/103192; U.S. 2002/65282; U.S. 2002/61860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Furumai et al., 2002; Hinnebusch et al., 2002; Mai et al., 2002; Vigushin et al., 2002; Gottlicher et al., 2001; Jung, 2001; Komatsu et al., 2001; Su et al., 2000.

E. MCIP

Another gene that is associated with heart failure and hypertrophy, primarily due to its tight association with and regulation by Calcineurin, is the human gene (DSCR1) encoding MCIP1, one of 50-100 genes that reside within a critical region of chromosome 21 (Fuentes et al., 1997; Fuentes et al., 1995), trisomy of which gives rise to the complex developmental abnormalities of Down syndrome, which include cardiac abnormalities and skeletal muscle hypotonia as prominent features (Epstein, 1995). ZAKI-4 was identified from a human fibroblast cell line in a screen for genes that are transcriptionally activated in response to thyroid hormone (Miyazaki et al., 1996).

MCIP1 directly binds and inhibits calcineurin, functioning as an endogenous feedback inhibitor of calcineurin activity. Overexpression of MCIP1 in the hearts of transgenic animals is anti-hypertrophic; MCIP1 attenuates in vivo models of both calcineurin-dependent hypertrophy (Rothermel et al., 2001) and pressure-overload-induced hypertrophy. (Hill et al., 2002). MCIP1 also acts as a substrate for phosphoryalation by MAPK and GSK-3, and calcineurin's phosphatase activity. Residues 81-177 of MCIP1 retain the calcineurin inhibitory action.

Binding of MCIP1 to calcineurin does not require calmodulin, nor does MCIP interfere with calmodulin binding to calcineurin. This suggests that the surface of calcineurin to which MCIP1 bindings does not include the calmodulin binding domain. In contrast, the interaction of MCIP1 with calcineurin is disrupted by FK506:FKBP or cyclosporin:cyclophylin, indicating that the surface of calcineurin to which MCIP1 binds overlaps with that required for the activity of immunosuppressive drugs.

MCIP, as well as all the aforementioned genes, each in and of themselves present enticing therapeutic targets for heart failure and hypertrophy. A major reason for the inventors interest in TRP channels is that these channels are potentially implicated in pathways and mechanisms that involve or recruit these genes. As such, treatment of heart failure or hypertrophy by inhibitin TRP channels would represent a major leap forward over the current methods available for treating patients suffering from these diseases.

IV. Methods of Treating Heart Failure and Cardiac Hypertrophy

A. Therapeutic Regimens for Heart Failure and Hypertrophy

Heart failure of some forms may curable and these are dealt with by treating the primary disease, such as anemia or thyrotoxicosis. Also curable are forms caused by anatomical problems, such as a heart valve defect. These defects can be surgically corrected. However, for the most common forms of heart failure—those due to damaged heart muscle—no known cure exists. Treating the symptoms of these diseases helps, and some treatments of the disease have been successful. The treatments attempt to improve patients' quality of life and length of survival through lifestyle change and drug therapy. Patients can minimize the effects of heart failure by controlling the risk factors for heart disease, but even with lifestyle changes, most heart failure patients must take medication, many of whom receive two or more drugs.

Several types of drugs have proven useful in the treatment of heart failure: Diuretics help reduce the amount of fluid in the body and are useful for patients with fluid retention and hypertension; and digitalis can be used to increase the force of the heart's contractions, helping to improve circulation. Results of recent studies have placed more emphasis on the use of ACE inhibitors (Manoria and Manoria, 2003). Several large studies have indicated that ACE inhibitors improve survival among heart failure patients and may slow, or perhaps even prevent, the loss of heart pumping activity (for a review see De Feo et al., 2003; DiBianco, 2003).

Patients who cannot take ACE inhibitors may get a nitrate and/or a drug called hydralazine, each of which helps relax tension in blood vessels to improve blood flow (Ahmed, 2003).

Heart failure is almost always life-threatening. When drug therapy and lifestyle changes fail to control its symptoms, a heart transplant may be the only treatment option. However, candidates for transplantation often have to wait months or even years before a suitable donor heart is found. Recent studies indicate that some transplant candidates improve during this waiting period through drug treatment and other therapy, and can be removed from the transplant list (Conte et al., 1998).

Transplant candidates who do not improve sometimes need mechanical pumps, which are attached to the heart. Called left ventricular assist devices (LVADs), the machines take over part or virtually all of the heart's blood-pumping activity. However, current LVADs are not permanent solutions for heart failure but are considered bridges to transplantation.

As a final alternative, there is an experimental surgical procedure for severe heart failure available called cardiomyoplasty. (Dumcius et al., 2003) This procedure involves detaching one end of a muscle in the back, wrapping it around the heart, and then suturing the muscle to the heart. An implanted electric stimulator causes the back muscle to contract, pumping blood from the heart. To date, none of these treatments have been shown to cure heart failure, but can at least improve quality of life and extend life for those suffering this disease.

As with heart failure, there are no known cures to hypertrophy. Current medical management of cardiac hypertrophy, in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn and Bristow, 1996). Other pharmaceutical agents that have been disclosed for treatment of cardiac hypertrophy include angiotensin II receptor antagonists (U.S. Pat. No. 5,604,251) and neuropeptide Y antagonists (WO 98/33791).

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

As can be seen from the discussion above, there is a great need for a successful treatment approach to heart failure and hypertrophy. In one embodiment of the present invention, methods for the treatment of cardiac hypertrophy or heart failure utilizing inhibitors of TRP channels are provided. For the purposes of the present application, treatment comprises reducing one or more of the symptoms of heart failure or cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of inhibitors of TRP channels may prevent cardiac hypertrophy and its associated symptoms from arising.

B. Pharmaceutical Inhibitors

TRP channels are a fairly recent focus of research, and as such only a few inhibitors of these channels have been characterized However, as the interest in these channels grows, the number of compounds that can be used to module TRPC activity will increase. The compound 2-aminoethoxy diphenyborane (2-ABP) has been shown to be a non-specific but potent inhibitor of non-voltage gated channels and is capable of inhibiting TRP and TRPC channels. (Schindle et al., 2002; Mai et al., 2002). Gysembergh et al. (1999) showed that both 2-ABP and D-myo-INS(1,4,5)P₃ can be used to treat the damage caused by and perhaps even prevent damage to the heart by myocardial infarction. Gadolinium has been shown to inhibit channel formation in DT40 chicken cells (Vazquez et al., 2003), as well as in HEK293 cells (Trebak et al., 2002), as has SKF 96365, a calcium channel inhibitor (Bennett et al., 2001). Anti-G(q/11) antibody, the PLC inhibitor U-73122, La³⁺ and flufanemate (both non specific cation channel inhibitors) have been shown to inhbit TRP channels in the stomach. (Lee et al., 2003). PPI, an Src family tyrosine kinse inhibitor, was shown to modulate the TRPM channel activity in kidney cells (Xu et al., 2003). Lanthanum, a Ca2+ permeable channel inhibitor, was shown to block calcium channel influx mediated by TRP channels by Machaty et al. (2002). Also, condensed cortical F-actin was shown to be capable of inhibiting activation of TRPC channels in HEK293 cells (Ma et al., 2000).

C. Antisense Constructs

An alternative approach to inhibiting TRPC is antisense. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Another general class of inhibitors is ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

E. RNAi

RNA interference (also referred to as “RNA-mediated interference” or RNAi) is another mechanism by which TRPC expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Treatment regimens would vary depending on the clinical situation. However, long term maintenance would appear to be appropriate in most circumstances. It also may be desirable treat hypertrophy with inhibitors of TRP channels intermittently, such as within brief window during disease progression.

F. Antibodies

In certain aspects of the invention, antibodies may find use as inhibitors or TRPCs. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

G. Combined Therapy

In another embodiment, it is envisioned to use an inhibitor of a TRP channel in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using an inhibitor of a TRP channel may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an inhibitor of TRPC, or the other agent will be desired. In this regard, various combinations may be employed. By way of illustration, where the inhibitor of a TRP channel is “A” and the other agent is “B,” the following permutations based on 3 and 4 total administrations are exemplary:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are likewise contemplated.

H. Adjunct Therapeutic Agents for Combination Therapy

Pharmacological therapeutic agents and methods of administration, dosages, etc., are well known to those of skill in the art (see for example, the “Physicians Desk Reference,” Goodman and Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In addition, it should be noted that any of the following may be used to develop new sets of cardiac therapy target genes as β-blockers were used in the present examples (see below). While it is expected that many of these genes may overlap, new gene targets likely can be developed.

1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, b-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemhorrage or an increased likelyhood of hemhorraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (amlodipine) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, g aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

8. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlommethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.

c. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

I. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

J. Drug Formulations and Routes for Administration to Patients

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures.

The previously mentioned formulations are all contemplated for treating patients suffering from heart failure or hypertrophy. The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

V. Screening Methods

The present invention further comprises methods for identifying inhibitors of TRP channel activity in cardiac cells that are useful in the prevention or treatment or reversal of cardiac hypertrophy or heart failure. These assays may comprise random screening of large libraries of candidate substances; alternatively, the assays may be used to focus on particular classes of compounds selected with an eye towards structural attributes that are believed to make them more likely to inhibit the function of a TRP channel.

To identify an inhibitor of a TRP channel, one generally will determine the function of a TRP channel in the presence and absence of the candidate substance. For example, a method generally comprises:
(a) providing a cardiomyocyte;
(b) contacting said cardiomyocyte with a candidate inhibitor substance; and
(c) measuring an activity mediated by a TRPC channel on said cardiomyocyte;
   wherein a decrease in cardiomyocyte TRPC channel activity, as compared to TRPC channel activity of an untreated cell, identifies the candidate substance as an inhibitor of cardiac TRPC channel activity.

Assays also may be conducted in isolated cells, organs, or in living organisms.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit the activity or cellular functions of a TRP channel. The candidate substance may be a protein or fragment thereof, a small molecule, or even a nucleic acid. It may prove to be the case that the most useful pharmacological compounds will be compounds that are structurally related to 2-ABP, listed elsewhere in this document. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration, or which may affect the function of various other molecules. In one approach, one would generate a three-dimensional structure for a target molecule, or a fragment thereof. This could be accomplished by x-ray crystallography, computer modeling, or by a combination of both approaches.

It also is possible to use antibodies to ascertain the structure of a target compound, activator, or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecular libraries that are believed to meet the basic criteria for useful drugs in an effort to “brute force” the identification of useful compounds. Screening of such libraries, including combinatorially-generated libraries (e.g., peptide libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small peptide test compounds are synthesized on a solid substrate, such as plastic pins or some other surface. Such peptides could be rapidly screening for their ability to bind and inhibit a TRP channel.

C. In Cyto Assays

The present invention also contemplates the screening of compounds for their ability to modulate TRP channel activity in cells. Various cell lines can be utilized for such screening assays, including cells specifically engineered for this purpose.

D. In Vivo Assays

In vivo assays involve the use of various animal models of heart disease, including transgenic animals, that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria, including but not limited to. Also, measuring toxicity and dose response can be performed in animals in a more meaningful fashion than in in vitro or in cyto assays.

VI. Vectors for Cloning, Gene Transfer and Expression

Within certain embodiments, expression vectors are employed to express various products including TRP channels, antisense molecules, ribozymes or interfering RNAs. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the native TRP channel promoter will be employed to drive expression of either the corresponding TRP channel gene, a heterologous TRP cannel gene, a screenable or selectable marker gene, or any other gene of interest.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodhourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al, 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al, 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al, 1982; |
| | Heavy metals | Haslinger et al., |
| | | 1985; |
| | | Searle et al., 1985; |
| | | Stuart et al., 1985; |
| | | Imagawa et al., |
| | | 1987, Karin et al., |
| | | 1987; Angel et al., |
| | | 1987b; McNeall |
| | | et al., 1989 |
| MMTV (mouse mammary | Glucocorticoids | Huang et al., 1981; |
| tumor virus) | | Lee et al., |
| | | 1981; Majors et al., |
| | | 1983; Chandler |
| | | et al., 1983; |
| | | Ponta et al., 1985 |
| | | Sakai et al., 1988 |
| β-Interferon | poly(rI)x | Tavernier et al., |
| | poly(rc) | 1983 |
| Adenovirus 5 E2 | E1A | Imperiale et al., |
| | | 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., |
| | | 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, |
| | Antigen | 1990a, 1990b |
| Proliferin | Phorbol Ester-TPA | Mordacq et al., |
| | | 1989 |
| Tumor Necrosis Factor | PMA | Hensel et al., 1989 |
| Thyroid Stimulating | Thyroid Hormone | Chatterjee et al., |
| Hormone αGene | | 1989 |

Of particular interest are muscle specific promoters, and more particularly, cardiac specific promoters. These include the myosin light chain-2 promoter (Franz et al., 1994; Kelly et al., 1995), the alpha actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the alpha7 integrin promoter (Ziober and Kramer, 1996), the brain natriuretic peptide promoter (LaPointe et al., 1995) and the alpha B-crystallin/small heat shock protein promoter (Gopal, 1995), alpha myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

VII. Preparing Antibodies Reactive with or Inhibitory to TRP Channels

In yet another aspect, the present invention contemplates an antibody that is immunoreactive or inhibitory to a TRP channel of the present invention, or any portion thereof. An antibody can be a polyclonal or a monoclonal antibody, it can be humanized, single chain, or even an Fab fragment. In a preferred embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to TRP channel-related antigen epitopes.

In general, both polyclonal, monoclonal, and single-chain antibodies against TRP channels may be used in a variety of embodiments. A particularly useful application of such antibodies is in purifying native or recombinant TRP channel, for example, using an antibody affinity column. The operation of all accepted immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified TRP channel, polypeptide or peptide or cell expressing high levels of TRP channels. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bu1; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

VIII. Definitions

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon the animal's survival. In use of animal models, the response of treated transgenic animals and untreated transgenic animals is compared using any of the assays described herein (in addition, treated and untreated non-transgenic animals may be included as controls). A compound which causes an improvement in any parameter associated with heart failure used in the screening methods of the instant invention may thereby be identified as a therapeutic compound.

The terms “compound” and “chemical agent” refer to any chemical entity, pharmaceutical, drug, and the like that can be used to treat or prevent a disease, illness, sickness, or disorder of bodily function. Compounds and chemical agents comprise both known and potential therapeutic compounds. A compound or chemical agent can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the terms “antagonist” and “inhibitor” refer to molecules, compounds, or nucleic acids which inhibit the action of a cellular factor that may be involved in heart failure or cardiac hypertrophy. Antagonists may or may not be homologous to these natural compounds in respect to conformation, charge or other characteristics. Thus, antagonists may be recognized by the same or different receptors that are recognized by an agonist. Antagonists may have allosteric effects which prevent the action of an agonist. Alternatively, antagonists may prevent the function of the agonist. In contrast to the agonists, antagonistic compounds do not result in pathologic and/or biochemical changes within the cell such that the cell reacts to the presence of the antagonist in the same manner as if the cellular factor was present. Antagonists and inhibitors may include proteins, nucleic acids, carbohydrates, or any other molecules which bind or interact with a receptor, molecule, and/or pathway of interest.

As used herein, the term “modulate” refers to a change or an alteration in a biological activity. Modulation may be an increase or a decrease in protein activity, a change in kinase activity, a change in binding characteristics, or any other change in the biological, functional, or immunological properties associated with the activity of a protein or other structure of interest. The term “modulator” refers to any molecule or compound which is capable of changing or altering biological activity as described above.

### IX. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

NRVM culture. For preparations of neonatal rat ventricular myocytes (NRVMs), hearts were removed from 10-20 newborn (1-2 days old) Sprague-Dawley rats. Isolated ventricles were pooled, minced and dispersed by three 20-minute incubations at 37° C. in Ads buffer (116 mM NaCl, 20 mM HEPES, 10 mM NaH2PO4, 5.5 mM glucose, 5 mM KCl, 0.8 mM MgSO4, pH 7.4) containing collagenase Type II (65 U/ml, Worthington) and pancreatin (0.6 mg/ml, GibcoBRL). Dispersed cells were applied to a discontinuous gradient of 40.5% and 58.5% (v/v) Percoll (Amersham Biosciences), centrifuged, and myocytes collected from the interface layer. Myocyte preparations were pre-plated in Dulbecco's modified Eagle's medium (DMEM, Cellgro), supplemented with 10% (v/v) fetal bovine serum (FBS, HyClone), 4 mM L-glutamine and 1% penicillin/streptomycin for 1 hr at 37° C. to reduce fibroblast contamination, then plated at a density of 2.5×10⁵ cells per well on 6-well tissue culture plates (or 10,000 cells/well on 96-well tissue culture plates) coated with a 0.2% (w/v) gelatin solution.

After 24 hrs in culture, myocyte preparations were transferred to serum-free maintenance medium (DMEM supplemented with 0.1% (v/v) Nutridoma (Roche), L-glutamine and penicillin/streptomycin). For infection with calcineurin adenovirus, NRVM were exposed to adenovirus at a multiplicity of infection (MOI) of 25 for 48 h prior to analysis. Where indicated, NRVM were treated with, phenylephrine (20 mM, Sigma) FBS (10%), or 2-APB (Cayman Chemical) for 48 h.

Gene-Chip Screening. RNA was extracted from unstimulated NRVM and hypertrophic NRVM exposed to phenylephrine (Trizol Reagent, GibcoBRL). RNA samples were converted to biotin-labeled cRNA and hybridized to Rat expression arrays (Affymetrix GeneChip). Arrays were then washed, scanned and quantitated as per manufacturer's instructions.

Western Blots. For protein sample preparation, cultured cells were lysed in extraction buffer (50 mM Tris, pH 7.5, 150 mM NaCl, 1% Triton X-100, 0.5% deoxycholic acid, 0.1% SDS) supplemented with protease inhibitors (1 mM AEBSF, 10 mg/ml aprotinin, 0.1 mM leupeptin, 2 mM EDTA). Left ventricle samples were ground under liquid nitrogen and solubilized in extraction buffer containing protease inhibitors. Homogenates were centrifuged 10 min at 4° C. at 16,000 g and supernatants recovered. Protein concentrations were determined by the bicinchoninic acid method (BCA Protein Assay, Pierce) with bovine serum albumin as a standard. Equivalent quantities of protein samples (10 mg/lane) were denatured in Laemmli buffer and resolved on Tris-glycine SDS-PAGE gels (4-20% acrylamide gradient, Invitrogen). Resolved proteins were transferred to nitrocellulose membranes, blocked in 5% nonfat dry milk, and probed with rabbit polyclonal primary antibody (diluted in TBST; 50 mM Tris, pH 7.5, 150 mM NaCl, 0.1% Tween-20) supplemented with 5% nonfat dry milk. Primary antibodies used include: anti-TRPC1 and TRPC3 (Alomone Labs) and anti-MCIP1 (Myogen, Inc). Membranes were washed, probed with a goat anti-rabbit horseradish peroxidase-conjugated secondary antibody (Southern Biotechnology Associates), and processed for enhanced chemiluminescence (SuperSignal reagent, Pierce). To verify equivalent protein loading, membranes were subsequently reprobed with a polyclonal rabbit antibody to the housekeeping gene IP90-calnexin. Densitometric analysis of immunoreactive band images was performed using a ChemiImager (Alpha Innotech).

Hypertrophy and Toxicity Assays. Primary hypertrophy endpoints for NRVM included quantitation of ANF secretion, total cellular protein and cell volume. ANF in media supernatants was quantitated by competitive ELISA using a monoclonal anti-ANF antibody (Biodesign) and a biotinylated ANF peptide (Phoenix Peptide). Total cellular protein was quantitated by standard Coomassie dye-binding assay; cells were lysed in protein assay reagent (BioRad) and absorbance at A595 was measured after 1 hr. For cell volume measurements, NRVM cultured in 6-well dishes were harvested by treatment with trypsin (Cellgro). After recovery by centrifugation, cell pellets were washed in PBS, resuspended in 10 ml IsoFlow electrolyte solution (Beckman-Coulter) and analyzed with a Z2 Coulter Particle Counter and Size Analyzer (Beckman-Coulter). Cytotoxicity was quantitated by measuring release of adenylate kinase (AK) from cultured NRVM into culture medium (ToxiLight kit, Cambrex).

### Example 2

### In Vivo Models

Trans-thoracic Aortic Banding (TAB). For chronic left thoracotomy and aortic ligation, male Sprague-Dawley rats (Harlan, Indianapolis, Ind.; 8-9 weeks of age, 200-225 g) were anesthetized with 5% isoflurane (v/v 100% O₂), intubated and maintained at 2.0% isoflurane with positive pressure ventilation. A left thoracotomy through the third intercostal space was performed and the descending thoracic aorta, 3-4 mm cranial to the intersection of the aorta and azygous vein was isolated. A segment of 5-0 silk suture was then positioned around the isolated aorta to function as a ligature. A blunted hypodermic needle (gauge determined by weight) was placed between the aorta and the suture to prevent complete aortic occlusion when the suture was tied. When tying was completed, the needle was removed from between the aorta and ligature, re-establishing flow through the vessel. The thorax was then closed and the pneumothorax evacuated. After 7 days of recovery, animals were sacrificed and left ventricular tissue processed for Western blot analysis as described above. Average heart weight to body weight ratios in banded versus sham-operated rats increased 22% at 1 week (data not shown).

Isoproterenol Infusion. For pharmacologic induction of hypertrophy in vivo, nine to ten-week-old male Sprague-Dawley rats were anesthetized via passive inhalation of 2.0% isoflurane. When a level of surgical anesthesia was reached, an osmotic minipump (Alzet model 2001, Alza Corp., Palo Alto, Calif.) containing either vehicle (0.1% ascorbic acid in 0.9% NaCl), or isoproterenol (4.8 mg/kg/d) was subcutaneously implanted into the back between the scapulae followed by closure with 3-0 silk sutures. After 4 days of recovery, animals were sacrificed and left ventricular tissue processed for Western blot analysis as described above. Average heart weight to body weight ratios in isoproterenol versus vehicle-infused rats increased 48% (data not shown).

SHHF Model. The SHHF-Mcc-facp rat (SHHF) is a genetic model that has been selectively bred for spontaneous hypertension and heart failure. The lean male SHHF rats used in this study were obtained from the colony at University of Colorado at Boulder. The onset of CHF was determined by the development of dyspnea, piloerection, cyanosis, ascites, pleural effusion, cold tail and extremities and necropsy examination of heart and lungs.

### Example 3

### Results

Transcriptomic Analysis of Hypertrophic Cardiomyocytes. The inventors performed a transcriptomic survey of genes that were differentially expressed in non-hypertrophic neonatal rat ventricular myocytes (NRVM) and myocytes stimulated to undergo hypertrophy with the adrenergic agonist phenylephrine (PE). RNA isolated from NRVM was labeled, hybridized to Affymetrix GeneChip Rat Expression Arrays, scanned and quantitated. A summary of some genes observed to be induced during phenylephrine-dependent hypertrophy are listed in the Table 4.

TABLE 4

| Gene | Fold upregulated by PE |
|---|---|
| Myosin heavy chain, embryonic | 18 |
| Brain natriuretic factor | 4 |
| Atrial natriuretic factor | 2 |
| MCIP1 | 2.5 |
| Alpha skeletal actin | 2 |
| Transient receptor potential channel TRPC3 | 18 |

As shown, expression of known hypertrophic markers was induced by phenylephrine, including: embryonic myosin heavy chain, brain and atrial natriuretic peptides, alpha skeletal actin, and the calcineurin-induced gene MCIP1. In addition, the inventors observed that mRNA expression of the non-voltage-gated cation channel TRPC3 increased 19-fold in hypertrophic cardiomyocytes. Increased expression of this channel has not previously been described in association with cardiomyocyte hypertrophy.

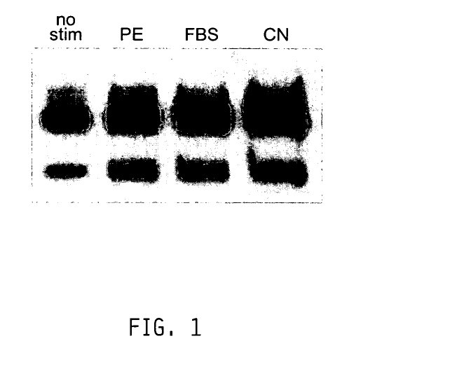

TRP Channel Expression in Hypertrophic Cardiomyocytes. To independently confirm that expression of TRPC3 protein was induced in hypertrophic cardiomyocytes, Western blot analysis with a TRPC3 antibody was performed on protein extracts from cultured NRVM exposed to three different hypertrophic stimuli: phenylephrine, fetal bovine serum or activated calcineurin (FIG. 1). All three hypertrophic stimuli significantly increased expression of TRPC3 channel protein in cardiomyocytes.

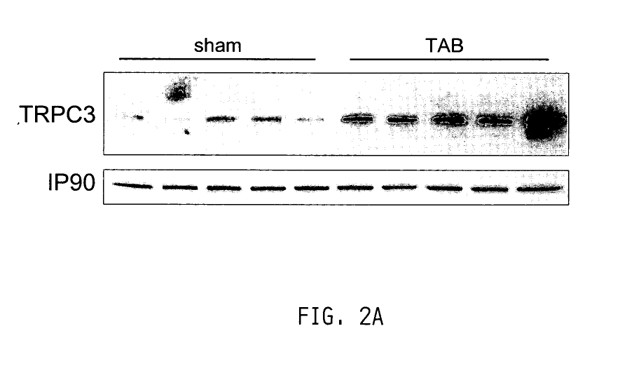

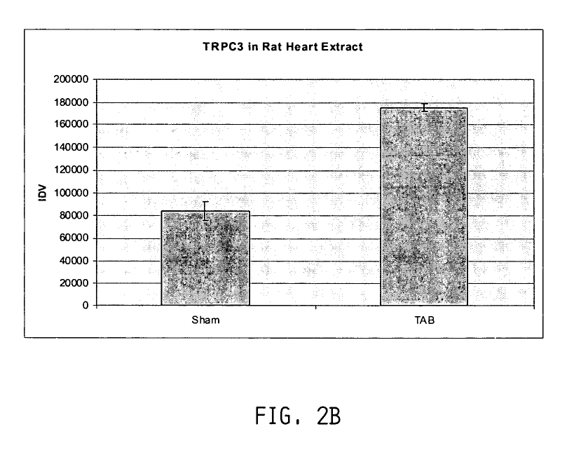

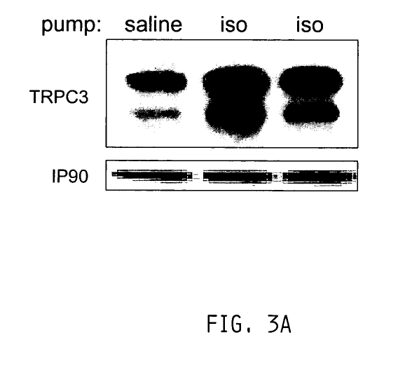

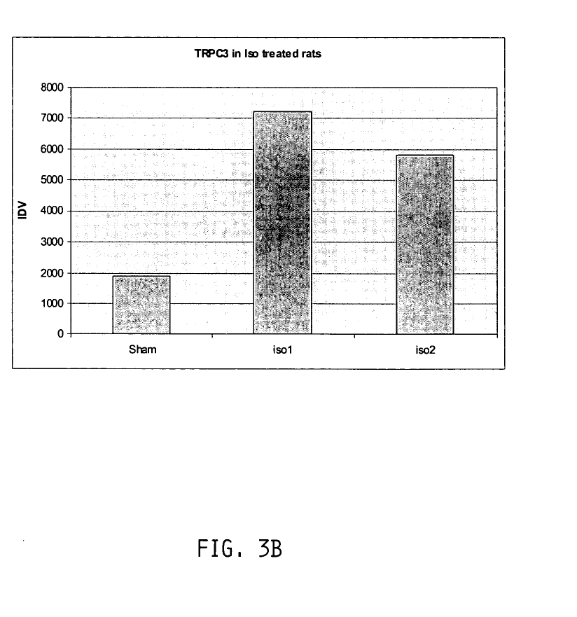

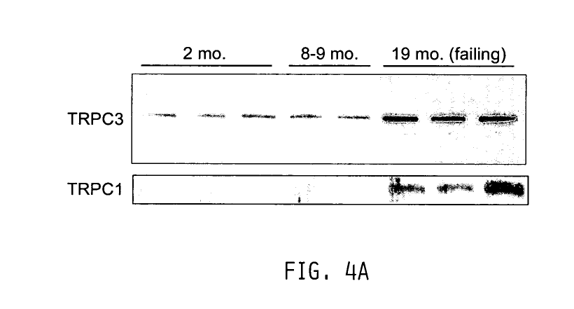

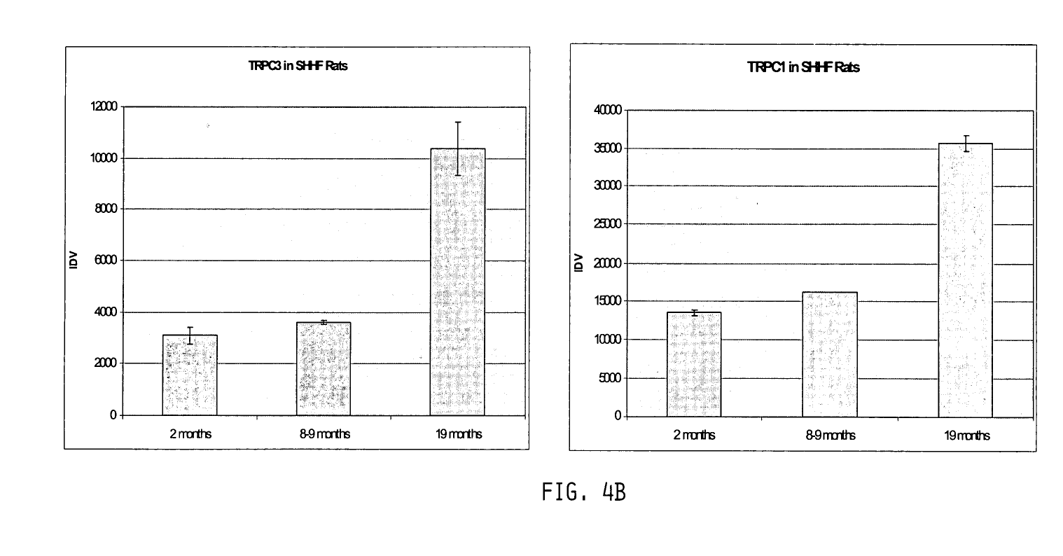

TRP Channel Expression in in vivo Models of Cardiac Hypertrophy and Heart Failure. The inventors next examined expression of TRP channel protein in three different in vivo rodent models of cardiac hypertrophy and heart failure: pressure overload induced by thoracic aortic banding (physiologic model), chronic isoproterenol infusion (pharmacologic model), and the spontaneously hypertensive heart failure rat (genetic model). As shown in FIG. 2, TRPC3 protein expression was induced approximately two-fold in left ventricles of animals subjected to thoracic aortic banding. Similarly, chronic isoproterenol infusion induced expression of ventricular TRPC3 protein approximately three-fold (FIG. 3). Finally, the inventors examined expression of TRPC3 and TRPC1 channels in a genetic model of dilated cardiomyopathy, the spontaneously hypertensive heart failure rat (SHHF). From 10-12 weeks of age, SHHF rats are hypertensive with systolic pressures ranging from 145-210 mm Hg. By the age of 16-22 months, lean males develop ventricular hypertrophy which progresses to dilated cardiomyopathy. As shown in FIG. 4, 2-month-old prehypertensive SHHF rats expressed relatively low levels of ventricular TRPC3 and TRPC1 protein. In contrast, ventricles from 19-month-old SHHF rats in heart failure expressed significantly more TRPC3 and TRPC1 protein (approximately three-fold and two-fold, respectively).

TRP Channel Antagonism in Cardiomyocytes. To evaluate the functional role TRP channels may play in the development of cardiac hypertrophy, the inventors examined whether the TRP channel antagonist 2-amino-ethoxydiphenyl borane (2-APB) could attenuate phenylephrine-induced cardiomyocyte hypertrophy as measured by atrial natriuretic factor expression, total cellular protein, cell volume and MCIP1 expression (an endogenous indicator of calcineurin activity). A known pharmacologic inhibitor of CRAC channel activity, 2-APB is thought to act by blocking signaling between the IP3 receptor and TRP channels (Shindl et al., 2002), although there is some evidence that channel antagonism may also occur directly (Gregory et al., 2001). Other calcium channels are not inhibited by 2-APB, including ryanodine receptors (Maruyama et al., 1997), voltage-gated calcium channels (Maruyama et al., 1997), arachidonic acid-activated calcium channels (Luo et al., 2001), S-nitrosylation-activated calcium channels (Van Rossum et al., 2000), calcium-activated chloride channels (Chorna-Ornan et al., 2001), or purinergic P2X receptor calcium channels.

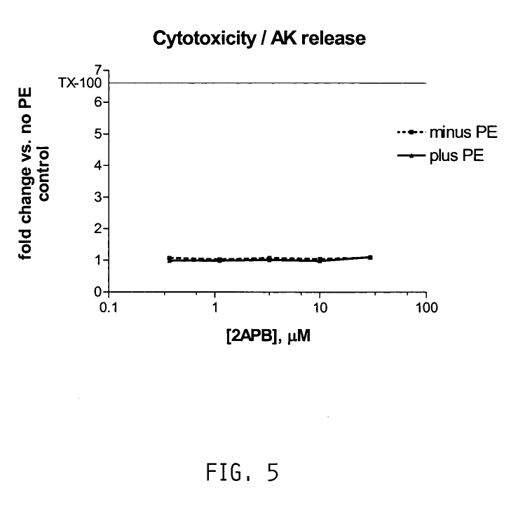

To assess potential cytotoxicity of 2-APB in cultured cardiomyocytes, NRVM were incubated for 48 hours with concentrations of 2-APB ranging from 0.3 to 30 μM. As shown in FIG. 5, no significant toxicity was observed at any concentration of 2-APB, as measured by adenylate kinase release (a standard method to determine cytotoxicity). Published concentrations for the in vitro use of 2-APB with other (non-myocyte) cell types are in the 30 to 75 μM range.

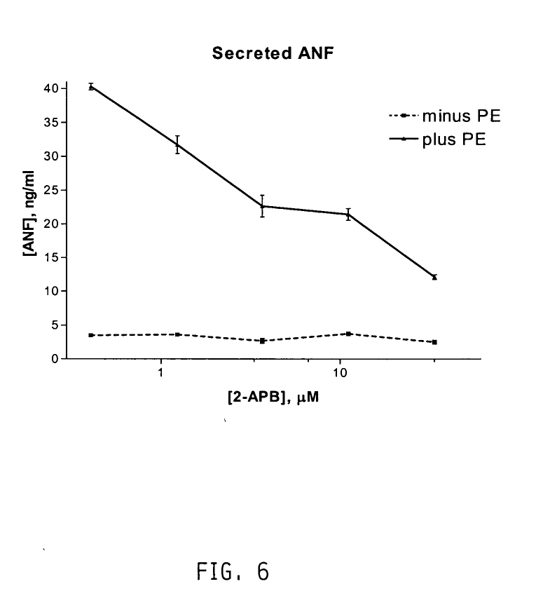

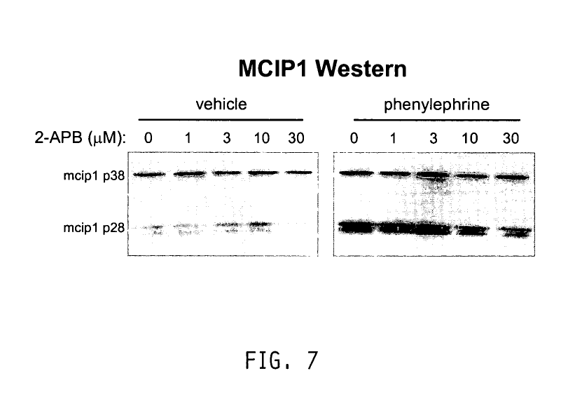

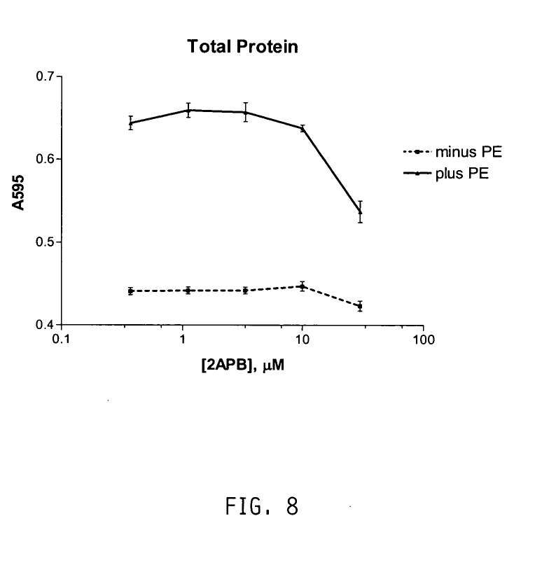

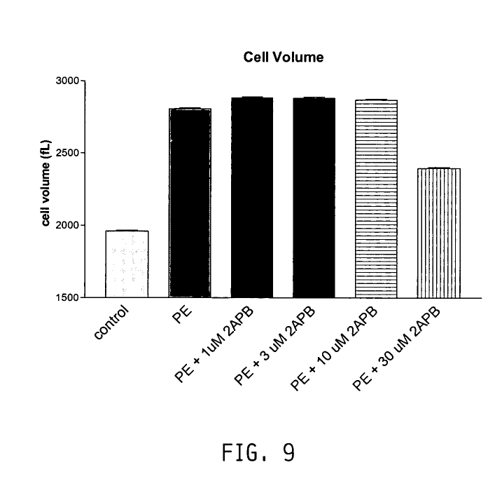

To determine whether 2-APB was capable of attenuating various indices of cardiac hypertrophy, NRVM were stimulated with phenylephrine along with increasing concentrations of 2-APB for a period of 48 hours. Secretion of atrial natriuretic factor is one of the most sensitive indicators of cardiomyocyte hypertrophy. As shown in FIG. 6, 2-APB effectively attenuated PE-dependent ANF secretion in a concentration-dependent fashion. Calcineurin is activated in a response to variety of hypertrophic stimuli, which in turn stimulates expression of the 28 kDa calcineurin-interacting protein MCIP1 (Yang et al., 2000). Phenylephrine strongly induced expression of 28 kDa MCIP1 protein, consistent with calcineurin activation (FIG. 7). Treatment with 2-APB attenuated induction of 28 kDa MCIP1 protein, consistent with inhibition of calcineurin signaling. Expression of a larger, 38 kDa calcineurin-independent MCIP1 isoform (Bush, unpublished observations) was unaffected by either PE or 2-APB. Slightly higher doses of 2-APB (10-30 micromolar) were also effective at inhibiting PE-dependent increases in total cellular protein (FIG. 8) and cell volume (FIG. 9).

Differential TRP Channel Expression in Three Rodent Models of Cardiac Hypertrophy. The inventors next performed Western blots to measure expression of TRPC3, TRPC1, TRPC4, TRPC5 and TRPC6 protein in three different in vivo rodent models of cardiac hypertrophy and heart failure: chronic isoproterenol infusion (pharmacologic model), pressure overload induced by thoracic aortic banding (physiologic model), and the spontaneously hypertensive heart failure rat (genetic model). Table 5 summarizing the densitometric analysis of TRPC isoform expression in the various models is represented below. Increased TRPC3 expression was a common feature of all three models. In contrast, increased expression of TRPC1, TRPC4 and TRPC5 was observed specifically with the SHHF, TAB and isoproterenol models, respectively. These observations indicate that distinct hypertrophic stimuli elicit different patterns of TRP channel expression. TRPC6 expression was not increased in any of the three rodent models.

TABLE 5

| Isoform | Rat iso | Rat TAB | SHHF |
|---|---|---|---|
| TRPC3 | ↑ | ↑ | ↑ |
| TRPC1 | [symbol] | [symbol] | ↑ |
| TRPC4 | [symbol] | ↑ | [symbol] |
| TRPC5 | ↑ | [symbol] | [symbol] |
| TRPC6 | [symbol] | [symbol] | [symbol] |

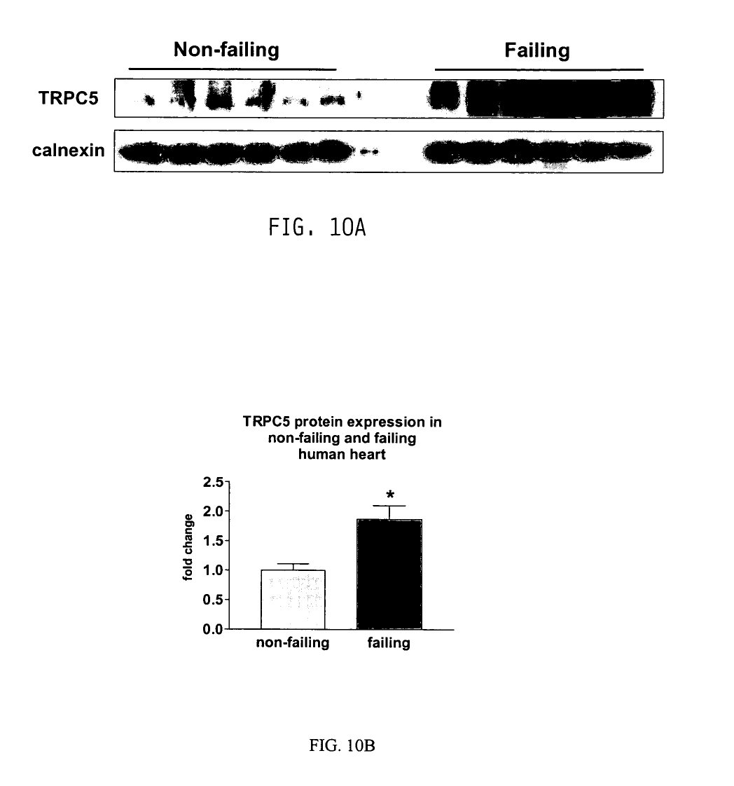

Increased TRPC5 Channel Expression in the Failing Human Heart. The inventors next examined expression of TRP channel protein expression in left ventricular tissue isolated from non-failing and failing (idiopathic dilated cardiomyopathy) human hearts. As shown in FIG. 10, expression of TRPC5 was increased by approximately two-fold in the failing human heart.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.
U.S. Pat. No. 4,166,452
U.S. Pat. No. 4,256,108
U.S. Pat. No. 4,265,874
U.S. Pat. No. 4,415,732
U.S. Pat. No. 4,458,066
U.S. Pat. No. 5,354,855
U.S. Pat. No. 5,604,251
U.S. Pat. No. 5,708,158
U.S. Pat. No. 5,795,715
U.S. Pat. No. 5,889,136
U.S. Pat. No. 6,372,957
U.S. Patent App. 2002/256221
U.S. Patent App. 2002/103192
U.S. Patent App. 2002/65282
U.S. Patent App. 2002/61860
Ahmed, Am. Geriatr. Soc., 51(1):123-126, 2003.
Angel et al., Mol. Cell. Biol., 7:2256, 1987a.
Angel et al., Cell, 49:729, 1987b.
Atchison and Perry, Cell, 46:253, 1986.
Atchison and Perry, Cell, 48:121, 1987.
Aus. Pat. No. 6,794,700
Aus. Pat. No. 9,013,101
Aus. Pat. No. 9,013,201
Aus. Pat. No. 9,013,401
Baichwal and Sugden, Gene Transfer, Kucherlapati (Ed.), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Bennet et al., Endocrinology, 142:1968-1974, 2001.
Benvenisty and Neshif, Proc. Natl. Acad. Sci. USA, 83:9551-9555, 1986.
Berkhout et al., Cell, 59:273-282, 1989.
Bhavsar et al., Genomics, 35(1): 11-23, 1996.
Blanar et al., EMBO J., 8:1139, 1989.
Bodine and Ley, EMBO J., 6:2997, 1987.
Boshart et al., Cell, 41:521, 1985.
Bosher et al., Nat. Cell. Biol., 2(2):E31-E36, 2000.
Bosze et al., EMBO J., 5(7):1615-1623, 1986.
Botinelli et al., Circ. Res. 82:106-115, 1997.
Braddock et al., Cell, 58:269, 1989.
Brand et al., J. Biochem. Cell. Biol., 29:1467-1470, 1997.
Bristow, Cardiology, 92:3-6, 1999.
Bulla and Siddiqui, J. Virol., 62:1437, 1986.
Campbell and Villarreal, Mol. Cell. Biol., 8:1993, 1988.
Campbell, In: Monoclonal Antibody Technology, Laboratory Techniques in Biochemistry and Molecular Biology, Vol. 13, Burden and Von Knippenberg (Eds.), 75-83, Amsterdam, Elseview, 1984.
Campere and Tilghman, Genes and Dev., 3:537, 1989.
Campo et al., Nature, 303:77, 1983.
Caplen et al., Gene, 252:95-105, 2000.
Celander and Haseltine, J. Virology, 61:269, 1987.
Celander et al., J. Virology, 62:1314, 1988.
Chandler et al., Cell, 33:489, 1983.
Chang et al., Hepatology, 14:124A, 1991.
Chang et al., Mol. Cell. Biol., 9:2153, 1989.
Chatterjee et al., Proc. Natl. Acad. Sci. USA, 86:9114, 1989.
Chein et al., Annu. Rev. Physiol., 55:77-95, 1993.
Chen and Okayama, Mol. Cell Biol., 7:2745-2752, 1987.
Choi et al., Cell, 53:519, 1988.
Chorna-Oman et al., J. Neurosci., 21:2622-2629, 2001.
Coffin, In: Virology, Fields et al. (Eds.), Raven Press, NY, 1437-1500, 1990.
Cohen et al., J. Cell. Physiol., 5:75, 1987.
Conte et al., J. Heart Lung Transplant., 17:679-685, 1998.
Cook et al., Cell, 27:487-496, 1981.
Costa et al., Mol. Cell. Biol., 8:81, 1988.
Couch et al., Am. Rev. Resp. Dis., 88:394-403, 1963.
Coupar et al., Gene, 68:1-10, 1988.
Crabtree and Olson, Cell, 109:S67-S79, 2002.
Cripe et al., EMBO J., 6:3745, 1987.
Culotta and Hamer, Mol. Cell. Biol., 9:1376, 1989.
Dandolo et al., J. Virology, 47:55-64, 1983.
De Feo et al., Ital. Heart J., 4:511-513, 2003.
De Villiers et al., Nature, 312(5991):242-246, 1984.
Deschamps et al., Science, 230:1174-1177, 1985.
DiBianco, Am. J. Med., 115:480-488, 2003.
Dolmetsch et al., Nature, 386:855-858, 1997.
Dubensky et al., Proc. Natl. Acad. Sci. USA, 81:7529-7533, 1984.
Dumcius et al., Medicina, 39:815-822, 2003.
Durand et al., Ann. Med., 27:311-317, 1995.
Edbrooke et al., Mol. Cell. Biol., 9:1908, 1989.
Edlund et al., Science, 230:912-916, 1985.
Eichhorn and Bristow, Circulation, 94:2285-2296, 1996.
Elbashir et al., EMBO, 20(23):6877-6888, 2001.
Emmel et al., Science, 246:1617-1620, 1989.
Epstein, In: The Metabolic and Molecular Bases of Inherited Disease: Down Syndrome (Trisomy 21), Scriver et al., (Eds), 7^{th} Ed., 1:749-794, McGraw-Hill, Inc., NY, 1995.
Eur. App. No. 0273085
Eur. Pat. No, 1,170,008.
Eur. Pat. No. 1,123,111.
Eur. Pat. No. 1,173,562.
Eur. Pat. No. 1,174,438.
Eur. Pat. No. 1,208,086.
Eur. Pat. No. 1,233,958.
Fechheimer, et al., Proc. Natl. Acad. Sci. USA, 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Fire et al., Nature, 391:806-811, 1998.
Foecking and Hofstetter, Gene, 45(1):101-105, 1986.
Forster and Symons, Cell, 49:211-220, 1987.
Fraley, et al., Proc Natl. Acad. Sci. USA, 76:3348-3352, 1979.
Franz et al., Cardoscience, 5(4):235-43, 1994.
Friedmann, Science, 244:1275-1281, 1989.
Fuentes, et al., Genomics, 44:358-361, 1997.
Fuentes, et al., Hum. Mol. Genet., 4:1935-1944, 1995.
Fujita et al., Cell, 49:357, 1987.
Furumai et al., Cancer Res., 62:4916-21, 2002.
Gao et al., J. Biol. Chem., 277:25748-55, 2002.
Gefter, et al., Somatic Cell Genet., 3:231-236, 1977.
Gerlach et al., Nature (London), 328:802-805, 1987.
Ghosh and Bachhawat, In: Liver Diseases, Targeted Diagnosis and Therapy Using Specific Receptors and Ligands. Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al, EMBO J., 6:3735, 1987.
Go et al., J. Clin. Invest., 95:888-894, 1995.
Godbout et al., Mol. Cell. Biol., 8:1169, 1988.
Goding, 1986, In: Monoclonal Antibodies: Principles and Practice, 2d ed., Academic Press, Orlando, Fla., 60-61, and 71-74, 1986.
Gomez-Foix et al, J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Goodman and Gilman's The Pharmacological Basis Of Therapeutics, Hardman et al. (Eds.), 10^{th} ed., 32:853-860; 35:891-893, 2001.
Gopal, Mol. Cell Biol., 5:1188-1190, 1985.
Gottlicher et al., EMBO J., 20:6969-78, 2001.
Graham and Prevec, In: Methods in Molecular Biology: Gene Transfer and Expression Protocol, Murray (Eds.), Humana Press, NJ, 7:109-128, 1991.
Graham and van der Eb, Virology, 52:456-467, 1973.
Graham et al., J. Gen. Virol., 36:59-72, 1977.
Greene et al., Immunology Today, 10:272, 1989
Gregory et al., Biochem J., 354:285-290, 2001.
Grishok et al., Science, 287:2494-2497, 2000.
Grosschedl and Baltimore, Cell, 41:885, 1985.
Grozinger et al., Proc. Natl. Acad. Sci. USA, 96:4868-4873, 1999.
Grunhaus and Horwitz, Seminar in Virology, 3:237-252, 1992.
Gruver et al., Endocrinology, 133:376-388, 1993.
Gysembergh et al., Am. J. Physiol., 277:H2458-H2469, 1999.
Han et al., Cancer Research, 60:6068-6074, 2000.
Harland and Weintraub, J. Cell Biol., 101: 1094-1099, 1985.
Harlow and Lane, Antibodies: A Laboratory Manual. Cold Spring Harbor Laboratory Press, Cold Spring harbor, NY, 553-612, 1988.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Natl. Acad. Sci. USA, 90:2812-2816, 1993.
Hill et al., J. Biol. Chem., 277:10251-10255, 2002.
Hinnebusch et al., J. Nutr., 132:1012-7, 2002.
Hirochika et al., J. Virol., 61:2599, 1987.
Hirsch et al., Mol. Cell. Biol., 10:1959, 1990.
Ho et al., J. Biol. Chem., 270:19898-19907, 1995.
Hoey et al., Immunity, 2:461-472, 1995.
Hoffmann et al., Bioconjugate Chem., 12:51-55, 2001.
Holbrook et al., Virology, 157:211, 1987.
Horlick and Benfield, Mol. Cell. Biol., 9:2396, 1989.
Horwich, et al., J. Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Japanese Appln. 2001/348340
Jayaraman and Marks, J. Boil. Chem., 275:6417-6420, 2000.
Jaynes et al., Mol. Cell. Biol., 8:62, 1988.
Johnson et al., Mol. Cell. Biol., 9:3393, 1989.
Jones and Shenk, Cell, 13:181-188, 1978.
Joyce, Nature, 338:217-244, 1989.
Jung et al., J. Med. Chem., 42:4669-4679, 1999.
Jung et al., Med. Chem. Lett., 7:1655-1658, 1997.
Jung, Curr. Med. Chem., 8:1505-11, 2001.
Kadesch and Berg, Mol. Cell. Biol., 6:2593, 1986.
Kaneda et al., Science, 243:375-378, 1989.
Kao et al., Genes Dev., 14:55-66, 2000.
Karin et al., Mol. Cell. Biol., 7:606, 1987.
Karin et al., Mol. Cell. Biol., 7:606, 1987.
Karlsson et al., EMBO J., 5:2377-2385, 1986.
Katinka et al., Cell, 20:393, 1980.
Kato et al., J. Biol. Chem., 266(6):3361-3364, 1991.
Kawamoto et al., Mol. Cell. Biol., 8:267, 1988.
Kelly et al., J. Cell Biol., 129(2):383-96, 1995.
Ketting et al., Cell, 99(2):133-141, 1999.
Kiledjian et al., Mol. Cell. Biol., 8:145, 1988.
Kim and Cook, Proc. Natl. Acad. Sci. USA, 84:8788-8792, 1987.
Kim et al., Oncogene, 18:2461-2470, 1999.
Kimura et al., Dev. Growth Differ., 39(3):257-65, 1997.
Kitazomo et al., J. Clinical Endoc. Metabol., 86(7):3430-3435, 2001.
Klamut et al., Mol. Cell. Biol., 10:193, 1990.
Klein et al., Nature, 327:70-73, 1987.
Koch et al., Mol. Cell. Biol., 9:303, 1989.
Kohler and Milstein, Eur. J. Immunol., 6:511-519, 1976.
Kohler and Milstein, Nature, 256:495-497, 1975.
Komastsu et al., Cancer Res., 61:4459-4466, 2001.
Kramer et al., Trends in Endoc. Metabolism, 12)7):294-300, 2001.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kudoh et al., Circ. Res., 80:139-146, 1997.
Kuhl et al., Cell, 50:1057, 1987.
Kunz et al., Nucl. Acids Res., 17:1121, 1989.
LaPoint et al., Hypertension, 27:715-722, 1995.
LaPointe, et al., J. Biol. Chem., 263(19):9075-8, 1988.
Larsen et al., Proc Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Am. J. Physiol. Gastrointest. Liver Physiol., 284:G604-G616, 2003.
Lee et al., Nature, 294:228, 1981.
Lee et al., Nucleic Acids Res., 12:4191-206, 1984.
Levinson et al., Nature, 295:79, 1982.
Levrero et al., Gene, 101: 195-202, 1991.
Levy et al., N. Engl. J. Med., 322:1561-1566, 1990.
Lin et al., Genetics, 153:1245-1256, 1999.
Lin et al., J. Clin. Invest., 97:2842-2848, 1996.
Lin et al., Mol. Cell. Biol., 10:850, 1990.
Luo et al., J. Biol. Chem., 276:20186-20189, 2001.
Luria et al., EMBO J., 6:3307,1987.
Lusky and Botchan, Proc. Natl. Acad. Sci. USA, 83:3609, 1986.
Lusky et al., Mol. Cell. Biol., 3:1108, 1983.
Ma et al., J. Biol. Chem., 277:6915-6922, 2002.
Macejak and Sarnow, Nature, 353:90-94, 1991.
Machaty et al., Biol. Reprod., 66:667-674, 2002.
Mai et al., J. Med. Chem., 45:1778-1784, 2002.
Majors and Varmus, Proc. Natl. Acad. Sci. USA, 80:5866, 1983.
Mann et al., Cell, 33:153-159, 1983.
Manoria and Manoria, J. Indian. Med. Assoc., 101(5):311-312, 2003.
Markowitz et al., J. Virol., 62:1120-1124, 1988.
Maruyama et al., J. Biochem., 122:498-505, 1997.
Massa et al., J. Med. Chem., 44:2069-2072, 2001.
Masuda et al., Mol. Cell. Biol., 15:2697-2706, 1995.
McCaffery et al., Science, 262:750-754, 1993.
McNeall et al., Gene, 76:81, 1989.
Merck Index, Thirteenth Edition
Michel and Westhof, J. Mol. Biol., 216:585-610, 1990.
Miksicek et al., Cell, 46:203, 1986.
Miyazaki, et al., J. Biol. Chem., 271:14567-14571, 1996.
Molkentin et al., Cell, 93:215-228, 1998.
Montgomery et al., Proc. Nat'l. Acad. Sci., 95:15502-15507, 1998.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Ng et al., Nuc. Acids Res., 17:601, 1989.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Northrop et al., Nature, 369:497-502, 1994.
Olson and Williams, Cell, 101:689-692, 2000a.
Olson and Williams, Bioassays, 22:510-519, 2000b.
Olson et al., Dev. Biol., 172(1):2-14, 1995.
Ondek et al., EMBO J., 6:1017, 1987.
Ornitz et al., Mol. Cell. Biol., 7:3466, 1987.
Palmiter and Solaro, Basic. Res. Cardiol., 92:63-74, 1997.
Palmiter et al., Nature, 300:611, 1982.
Park et al., J. Biol. Chem., 271:20914-20921, 1996.
Paskind et al., Virology, 67:242-248, 1975.
PCT Appl. WO 00/44914
PCT Appl. WO 01/14581
PCT Appl. WO 01/18045
PCT Appl. WO 01/36646
PCT Appl. WO 01/38322
PCT Appl. WO 01/42437
PCT Appl. WO 01/68836
PCT Appl. WO 01/70675
PCT Appl. WO 02/26696
PCT Appl. WO 02/26703
PCT Appl. WO 02/30879
PCT Appl. WO 02/46129
PCT Appl. WO 02/46144
PCT Appl. WO 02/50285
PCT Appl. WO 02/51842
PCT Appl. WO 84/03564
PCT Appl. WO 98/33791
PCT Appl. WO 99/32619
Pech et al., Mol. Cell. Biol., 9:396, 1989.
Pelletier and Sonenberg, Nature, 334:320-325, 1988.
Perales, et al., Proc. Natl. Acad. Sci. USA, 91(9):4086-4090, 1994.
Perez-Stable and Constantini, Mol. Cell. Biol., 10:1116, 1990.
Physicians Desk Reference.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reinhold-Hurek and Shub, Nature, 357:173-176, 1992.
Reisman and Rotter, Mol. Cell. Biol., 9:3571, 1989.
Remington's Pharmaceutical Sciences and The Merck Index, 11th Edition.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A Survey of Molecular Cloning Vectors and Their Uses, Rodriguez et al. (Eds.), Stoneham: Butterworth, 467-492, 1988.
Ripe et al., Mol. Cell. Biol., 9:2224, 1989.
Rippe, et al., Mol. Cell Biol., 10:689-695, 1990.
Rittling et al., Nuc. Acids Res., 17:1619, 1989.
Rooney et al., EMBO J., 13:625-633, 1994.
Rosen et al., Cell, 41:813, 1988.
Rosenfeld, et al., Cell, 68:143-155, 1992.
Rosenfeld, et al., Science, 252:431-434, 1991.
Rothermel et al., Proc. Natl. Acad. Sci. USA, 98:3328-3333, 2001.
Roux et al., Proc. Natl. Acad. Sci. USA, 86:9079-9083, 1989.
Sadoshima and Izumo, Circ. Res., 73:424-438, 1993.
Sadoshima et al., Annu. Rev. Physiol., 59:551-571, 1997.
Sadoshima et al., Cell, 75:977-984, 1993.
Sakai et al., Genes and Dev., 2:1144, 1988.
Sarver et al., Science, 247:1222-1225, 1990.
Satake et al., J. Virology, 62:970, 1988.
Saunders et al., Cancer Res., 59-399-409, 1999.
Scanlon et al., Proc. Natl. Acad. Sci. USA, 88:10591-10595, 1991.
Schaffner et al., J. Mol. Biol., 201:81, 1988.
Schindl et al., J. Biol Chem, 277:26950-26958, 2002.
Searle et al., Mol. Cell. Biol., 5:1480, 1985.
Sharp and Marciniak, Cell, 59:229, 1989.
Sharp et al., Science, 287:2431-2433, 2000.
Sharp, P. A., Genes. Dev., 13:139-141, 1999.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stemmer and Klee, Biochemistry, 33:6859-6866, 1994.
Stephens and Hentschel, Biochem. J., 248:1, 1987.
Stratford-Perricaudet and Perricaudet, In: Human Gene Transfer, Cohen-Haguenauer and Boiron (Eds.), John Libbey Eurotext, France, 51-61, 1991.
Stuart et al., Nature, 317:828, 1985.
Su et al., Cancer Res., 60:3137-3142, 2000.
Su et al., Eur. J. Biochem., 230:469-474, 1995.
Sullivan and Peterlin, Mol. Cell. Biol., 7:3315, 1987.
Swartzendruber and Lehman, J. Cell. Physiology, 85:179, 1975.
Tabara et al., Cell, 99(2): 123-132, 1999.
Takahashi et al., Antibiotics, 49:453, 1996.
Takebe et al., Mol. Cell. Biol., 8:466, 1988.
Taunton et al., Science, 272:371, 1996.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Tong et al., Nucleic Acids Res., 30:1114-23, 2002.
Top et al., J. Infect. Dis., 124:155-160, 1971.
Trebak et al., J. Biol. Chem., 277:21617-21623, 2002.
Treisman, Cell, 42:889, 1985.
Tronche et al., Mol. Biol. Med., 7:173, 1990.
Trudel and Constantini, Genes and Dev. 6:954, 1987.
Tur-Kaspa, et al., Mol. Cell Biol., 6:716-718, 1986.
Tyndell et al., Nuc. Acids. Res., 9:6231, 1981.
Van den Wyngaert et al., FEBS Lett., 478:77-83, 2000.
Van Rossum et al., J. Biol. Chem., 275:28562-28568, 2000.
Vandebrouck et al., J. Cell. Biol., 158:1089-1096, 2002.
Vannice and Levinson, J. Virology, 62:1305, 1988.
Varmus et al., Cell, 25:23-36, 1981.
Vasquez et al., J. Biol. Chem., 278:21649-21654, 2003.
Vasseur et al., Proc Natl. Acad. Sci. U.S.A., 77:1068, 1980.
Venneken et al., Cell Calcium, 31:253-264, 2002.
Vigushin et al., Anticancer Drugs, 13:1-13, 2002.
Vigushin et al., Cancer Res., 5S, 1999.
Vigushin et al., Clinical Cancer Res., 7:971-976, 2001.
Vikstrom and Leinwand, Curr. Opin. Cell Biol., 8:97-105, 1996.
Wagner, et al., Proc. Nat'l Acad. Sci. USA 87(9):3410-3414, 1990.
Wang and Calame, Cell, 47:241, 1986.
Watkins et al., Hum. Mol. Genet., 4:1721-1727, 1995.
Weber et al., Cell, 36:983, 1984.
Weinberger et al. Mol. Cell. Biol., 8:988, 1984.
Wincott et al., Nucleic Acids Res., 23:2677-2684, 1995.
Winoto and Baltimore, Cell 59:649, 1989.
Wolfe et al., Nature, 385:172-176, 1997.
Wong et al., Gene, 10:87-94, 1980.
Workman and Kingston, Annu. Rev. Biochem., 67:545-579, 1998.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Xu et al., J. Biol. Chem., 278:11520-11527, 2003.
Yamano et al., Amer. Soc. Gene Ther., 2000.
Yamauchi-Takihara, et al., Proc. Natl. Acad. Sci. USA, 86(10):3504-3508, 1989.
Yamazaki et al., Circulation, 95:1260-1268, 1997.
Yang et al., Circ. Res., 87:E61-E68, 2000.
Yang, et al., Proc. Natl. Acad. Sci. USA, 87:9568-9572, 1990.
Young et al., Handbook of Applied Therapeutics, 7.1-7.12 and 9.1-9.10, 1989.
Yue et al., Nature, 410:705-709, 2001.
Yutzey et al. Mol. Cell. Biol., 9:1397,1989.
Zelenin et al., FEBS Lett., 280:94-96, 1991.
Zhou et al., Proc. Natl. Acad. Sci., 98:10572-10577,2001.
Ziober and Kramer, J. Bio. Chem., 271(37):22915-22, 1996.
Zou et al., J. Biol. Chem., 271:33592-33597,1996.

1. A method of treating cardiac hypertrophy or heart failure comprising:
(a) identifying a patient having cardiac hypertrophy or heart failure; and
(b) administering to said patient an inhibitor of a TRP channel.

2. The method of claim 1, wherein said inhibitor inhibits a TRPC channel.

3. The method of claim 2, wherein said inhibitor inhibits one or more of TRPC1, TRPC3, TRPC4, TRPC5 or TRPC6.

4. The method of claim 1, wherein said inhibitor is selected from the group consisting of an antibody, an RNAi, a ribozyme, a peptide, a small molecule, an antisense molecule, 2-ABP, D-myoI-INS(1,4,5)P₃, gadolinium, Anti-G(q/11) antibody, U-73122, La³⁺, flufanemate, PPI, lanthanum, or condensed cortical F-actin.

5. The method of claim 4, wherein the antibody is a monoclonal, polyclonal or humanized antibody, an Fab fragment, or a single chain antibody.

6. The method of claim 1, wherein administering comprises intravenous administration of said inhibitor.

7. The method of claim 1, wherein administering comprises oral, transdermal, sustained release, suppository, or sublingual administration of said inhibitor.

8. The method of claim 1, further comprising administering to said patient a second therapeutic regimen.

9. The method of claim 8, wherein said second therapeutic regimen is selected from the group consisting of a beta blocker, an iontrope, diuretic, ACE-I, AII antagonist, histone deacetylase inhibitor, and Ca(++)-blocker.

10. The method of claim 8 wherein said second therapeutic regimen is administered at the same time as said inhibitor.

11. The method of claim 8, wherein said second therapeutic regimen is administered either before or after said inhibitor.

12. The method of claim 1, wherein treating comprises improving one or more symptoms of cardiac hypertrophy.

13. The method of claim 12, wherein said one or more symptoms comprises increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, or wall tension, quality of life, disease-related morbidity and mortality.

14. The method of claim 1, wherein treating comprises improving one or more symptoms of heart failure.

15. The method of claim 14, wherein one or more symptoms comprises progressive remodeling, ventricular dilation, decreased cardiac output, impaired pump performance, arrhythmia, fibrosis, necrosis, energy starvation, and apoptosis.

16. A method of preventing cardiac hypertrophy or heart failure comprising:
(a) identifying a patient at risk for cardiac hypertrophy or heart failure; and
(b) administering to said patient an inhibitor of a TRP channel.

17. The method of claim 16, wherein said TRP channel is a TRPC channel.

18. The method of claim 17, wherein said TRPC channel is one or more of TRPC1, TRPC3, TRPC4, TRPC5 or TRPC6.

19. The method of claim 16, wherein administering comprises intravenous administration of said TRP channel inhibitor.

20. The method of claim 19, wherein administering comprises oral, transdermal, ustained release, suppository, or sublingual administration.

21. The method of claim 16, wherein the patient at risk may exhibit one or more of long standing uncontrolled hypertension, uncorrected valvular disease, chronic angina and/or recent myocardial infarction.

22. The method of claim 16, wherein said inhibitor of a TRP channel consists of an antibody, an RNAi, a ribozyme, a peptide, a small molecule, an antisense molecule, 2-ABP, D-myoI-INS(1,4,5)P₃, gadolinium, Anti-G(q/11) antibody, U-73122, La³⁺, flufanemate, PPI, lanthanum, or condensed cortical F-actin.

23. The method of claim 4, wherein the antibody is a monoclonal, polyclonal or humanized antibody, an Fab fragment, or a single chain antibody.

24. A method of identifying an inhibitor of cardiac TRPC channel activity comprising:
(a) providing a cardiomyocyte;
(b) contacting said cardiomyocyte with a candidate inhibitor substance; and
(c) measuring an activity mediated by a TRPC channel on said cardiomyocyte;
wherein a decrease in cardiomyocyte TRPC channel activity, as compared to TRPC channel activity of an untreated cell, identifies the candidate substance as an inhibitor of cardiac TRPC channel activity.

25. The claim of 23, wherein said activity mediated by TRPC channel comprises calcium flux, calcineurin activity, MCIP protein levels, MCIP RNA levels, or NF-AT3 mediated gene expression.

26. The method of claim 24, wherein said TRPC channels are located in intact cells, either endogenously or by induced over-expression.

27. The method of claim 24, wherein said cardiomyocytes are neonatal rat ventricular myocytes.

28. The method of claim 24, wherein said cardiomyocytes are located in an intact heart.

29. The method of claim 28, wherein said heart is a human heart.

30. A method of identifying an inhibitor of heart failure or hypertrophy comprising:
(a) providing a TRP channel inhibitor;
(b) treating a myocyte with said TRP channel inhibitor; and
(c) measuring the expression of one or more cardiac hypertrophy or heart failure parameters,
wherein a change in said one or more cardiac hypertrophy or heart failure parameters, as compared to one or more cardiac hypertrophy parameters in a myocyte not treated with said TRP channel inhibitor, identifies said TRP channel inhibitor as an inhibitor of heart failure or cardiac hypertrophy.

31. The method of claim 30, wherein said myocyte is subjected to a stimulus that triggers a hypertrophic response in said one or more cardiac hypertrophy parameters.

32. The method of claim 31, wherein said stimulus is expression of a transgene.

33. The method of claim 31, wherein said stimulus is treatment with a chemical agent.

34. The method of claim 33, wherein said one more cardiac hypertrophy parameters comprises the expression level of one or more target genes in said myocyte, wherein expression level of said one or more target genes is indicative of cardiac hypertrophy.

35. The method of claim 34, wherein said one or more target genes is selected from the group consisting of ANF, α-MyHC, β-MyHC, α-skeletal actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen.

36. The method of claim 30, wherein the expression level is measured using a reporter protein coding region operably linked to a target gene promoter.

37. The method of claim 36, wherein said reporter protein is luciferase, β-gal, or green fluorescent protein.

38. The method of claim 30, wherein the expression level is measured using hybridization of a nucleic acid probe to a target mRNA or amplified nucleic acid product.

39. The method of claim 30, wherein said one or more cardiac hypertrophy parameters comprises one or more aspects of cellular morphology.

40. The method of claim 39, wherein said one or more aspects of cellular morphology comprises sarcomere assembly, cell size, cellular fusion, or cell contractility.

41. The method of claim 30, wherein said myocyte is an isolated myocyte.

42. The method of claim 30, wherein said myocyte is comprised in isolated intact tissue.

43. The method of claim 30, wherein said myocyte is a cardiomyocyte.

44. The method of claim 43, wherein said cardiomyocyte is a neonatal rat ventricular myocyte.

45. The method of claim 44, wherein said cardiomyocyte is located in vivo in a functioning intact heart muscle.

46. The method of claim 45, wherein said functioning intact heart muscle is subjected to a stimulus that triggers heart failure or a hypertrophic response in one or more cardiac hypertrophy parameters.

47. The method of claim 46, wherein said stimulus is aortic banding, rapid cardiac pacing, induced myocardial infarction, osmotic minipump, or transgene expression.

48. The method of claim 47, wherein said one or more cardiac hypertrophy parameters comprises right ventricle ejection fraction, left ventricle ejection fraction, ventricular wall thickness, heart weight/body weight ratio, or cardiac weight normalization measurement.

49. The method of claim 30, wherein said one or more cardiac hypertrophy parameters comprises total protein synthesis.


SEQUENCE LISTING (4 sequences)

SEQ ID NO: 1 - DNA, 4085 bp, Homo sapiens
  CDS (138)..(2417)
ccgggcctcgagccgaggcagcagtgggaacgactcatcctttttccagccctggggcgtggctggggtcggggtcggggtcggggccggtgggggccccgcccccgtctcctggcctgcccccttcatgggccgcgatgatggcggccctgtacccgagcacggacctctcgggcgcctcctcctcctccctgccttcctctccatcctcttcctcgccgaacgaggtgatggcgctgaaggatgtgcgggaggtgaaggaggagaatacgctgaatgagaagcttttcttgctggcgtgcgacaagggtgactattatatggttaaaaagattttggaggaaaacagttcaggtgacttgaacataaattgcgtagatgtgcttgggagaaatgctgttaccataactattgaaaacgaaaacttggatatactgcagcttcttttggactacggttgtcagaaactaatggaacgaattcagaatcctgagtattcaacaactatggatgttgcacctgtcattttagctgctcatcgtaacaactatgaaattcttacaatgctcttaaaacaggatgtatctctacccaagccccatgcagttggctgtgaatgcacattgtgttctgcaaaaaacaaaaaggatagcctccggcattccaggtttcgtcttgatatatatcgatgtttggccagtccagctctaataatgttaacagaggaggatccaattctgagagcatttgaacttagtgctgatttaaaagaactaagtcttgtggaggtggaattcaggaatgattatgaggaactagcccggcaatgtaaaatgtttgctaaggatttacttgcacaagcccggaattctcgtgaattggaagttattctaaaccatacgtctagtgacgagcctcttgacaaacggggattattagaagaaagaatgaatttaagtcgtctaaaacttgctatcaaatataaccagaaagagtttgtctcccagtctaactgccagcagttcctgaacactgtttggtttggacagatgtcrggttaccgacgcaagcccacctgtaagaagataatgactgttttgacagtaggcatcttttggccagttttgtcactttgttatttgatagctcccaaatctcagtttggcagaatcattcacacaccttttatgaaatttatcattcatggagcatcatatttcacatttctgctgttgcttaatctatactctcttgtctacaatgaggataagaaaaacacaatggggccagcccttgaaagaatagactatcttcttattctgtggattattgggatgatttggtcagacattaaaagactctggtatgaagggttggaagactttttagaagaatctcgtaatcaactcagttttgtcatgaattctctttatttggcaacctttgccctcaaagtggttgctcacaacaagtttcatgattttgctgatcggaaggattgggatgcattccatcctacactggtggcagaagggctttttgcatttgcaaatgttctaagttatcttcgtctcttttttatgtatacaaccagctctatcttgggtccattacagatttcaatgggacagatgttacaagattttggaaaatttcttgggatgtttcttcttgttttgttttctttcacaattggactgacacaactgtatgataaaggatatacttcaaaggagcagaaggactgtgtaggcatcttctgtgaacagcaaagcaatgataccttccattcgttcattggcacctgctttgctttgttctggtatattttctccttagcgcatgtggcaatctttgtcacaagatttagctatggagaagaactgcagtcctttgtgggagctgtcattgttggtacatacaatgtcgtggttgtgattgtgcttaccaaactgctggtggcaatgcttcataaaagctttcagttgatagcaaatcatgaagacaaagaatggaagtttgctcgagcaaaattatggcttagctactttgatgacaaatgtacgttacctccacctttcaacatcattccctcaccaaagactatctgctatatgattagtagcctcagtaagtggatttgctctcatacatcaaaaggcaaggtcaaacggcaaaacagtttaaaggaatggagaaatttgaaacagaagagagatgaaaactatcaaaaagtgatgtgctgcctagtgcatcgttacttgacttccatgagacagaagatgcaaagtacagatcaggcaactgtggaaaatctaaacgaactgcgccaagatctgtcaaaattccgaaatgaaataagggatttacttggctttcggacttctaaatatgctatgttttatccaagaaattaaccattttctaaatcatggagcgaataattttcaataacagatccaaaagactatattgcataacttgcaatgaaattaatgagatatatattgaaataaagaattatgtaaaagccattctttaaaatatttatagcataaatatatgttatgtaaagtgtgtatatagaattagttttttaaaccttctgttagtggctttttgcagaagcaaaacagattaagtagatagattttgttagcatgctgcttggttttcttacttagtgctttaaaatgtttttttttatgtttaagaggggcagttataaatggacacattgcccagaatgttttgtaaaatgaagaccagcaaatgtaggctgatctccttcacaggatacacttgaaatatagaagttatgttttaaatatctctgttttaggagttcacatatagttcagcatttattgtttaggagtataattttattttatctaaaataatagtctattttttcttttgtattttgttataatcttaagcaacaaagaaaaaaccctaatatttgaatctatttatgtctttcaatttaaattcacttcagtttttgttattgtaatatatttacttttacatggttataatcactttatatttttaatgtttttttcacttaatattttatatatacatttccatgtattgatgtagttagtccacatttaaatttttatagaattatatagtttttgaaaaatacagtcagtagatgttttattttttagctattcagttatgtttataagtttgcatagctacttctcgacatttggtttgttttaatttttttgtatcataatagtcctatttttttttcaagttggagtgaatgtttttagttttaagatagataggagacacttttttatcacatgtagtcacaacctgttttgtttttgtaaaacataggaagtctctttaatgcaatgatttgttttatatttggactaaggttcttgagcttatctcccaaggtactttccataatttaacacagcttctataaaagtgacttcatgcttacttgtggatcattcttgctgcttaagatgaaaagcattggttttttaaaattagagaataaaatatgtatttaaatttttggtgtgttcacataaagggatgtagctaaaatgttttcataggctattatatattctcgcagcatttccagttaagaggatattaggtatataattctcttcttaaccgaatgtcagatggtcttacgccacagggtgcaggtaacccttggtctgtaagcaccaccgatccagggatcattgtctaaataggttactattgtttgtttcatcttgcttttgcatttttattttttaatttccaaattttaagtgttccctctttggggcaaattcttataaaaatgtttattgtaaagttatatattttgtctacgatgggattatgcacttcccaattgggattttacatctggatttttagtcattctaaaaaacacctaattattaaaacatttatagagtgcctactgtatgcatgagttgagttgcttctgaggtacattttgaatgacagcatattgtaagaaaaaaaaaggtgaataaaatttgacattagattataaaaaaaaaaaggaattc

SEQ ID NO: 2 - protein, 759 aa, Homo sapiens
  MOD RES (306): X = anything
MMAALYPSTDLSGASSSSLPSSPSSSSPNEVMALKDVREVKEENTLNEKLFLLACDKGDYYMVKKILEENSSGDLNINCVDVLGRNAVTITIENENLDILQLLLDYGCQKLMERIQNPEYSTTMDVAPVILAAHRNNYEILTMLLKQDVSLPKPHAVGCECTLCSAKNKKDSLRHSRFRLDIYRCLASPALIMLTEEDPILRAFELSADLKELSLVEVEFRNDYEELARQCKMFAKDLLAQARNSRELEVILNHTSSDEPLDKRGLLEERMNLSRLKLAIKYNQKEFVSQSNCQQFLNTVWFGQMXGYRRKPTCKKIMTVLTVGIFWPVLSLCYLIAPKSQFGRIIHTPFMKFIIHGASYFTFLLLLNLYSLVYNEDKKNTMGPALERIDYLLILWIIGMIWSDIKRLWYEGLEDFLEESRNQLSFVMNSLYLATFALKVVAHNKFHDFADRKDWDAFHPTLVAEGLFAFANVLSYLRLFFMYTTSSILGPLQISMGQMLQDFGKFLGMFLLVLFSFTIGLTQLYDKGYTSKEQKDCVGIFCEQQSNDTFHSFIGTCFALFWYIFSLAHVAIFVTRFSYGEELQSFVGAVIVGTYNVVVVIVLTKLLVAMLHKSFQLIANHEDKEWKFARAKLWLSYFDDKCTLPPPFNIIPSPKTICYMISSLSKWICSHTSKGKVKRQNSLKEWRNLKQKRDENYQKVMCCLVHRYLTSMRQKMQSTDQATVENLNELRQDLSKFRNEIRDLLGFRTSKYAMFYPRN

SEQ ID NO: 3 - DNA, 3448 bp, Homo sapiens
  CDS (425)..(2971)
attaaccttctcttagtcttcaacctaagtacttgaatgtcaagtaccctccaaccctcaatgtcccaagacttttaagagcggaaggtaccgatgagttccatcctttactagggtcaccaaggaaggcatgggtatatggaaatttttattattattccatctgaatatcattttctagagaataggagcttttgttctgaagggctgccggcttccttctgggatctagcagccagggttagatcacaggtgtcactttcaggcgagtagttagcaacggtatcgctagcaactgagccgacccctgcagccagaggtttgcagtgggtagtgtgtattccagaaagggccctgacatgtgaaaggaaggaatgtgccctaatattctacagttgttttatcgttgctactgattaggtccatggagggaagcccatccctgagacgcatgacagtgatgcgggagaagggccggcgccaggctgtcaggggcccggccttcatgttcaatgaccgcggcaccagcctcaccgccgaggaggagcgcttcctcgacgccgccgagtacggcaacatcccagtggtgcgcaagatgctggaggagtccaagacgctgaacgtcaactgcgtggactacatgggccagaacgcgctgcagctggctgtgggcaacgagcacctggaggtgaccgagctgctgctcaagaaggagaacctggcgcgcattggcgacgccctgctgctcgccatcagcaagggctacgtgcgcatcgtagaggccatcctcaaccaccctggcttcgcggccagcaagcgtctcactctgagcccctgtgagcaggagctgcaggacgacgacttctacgcttacgacgaggacggcacgcgcttctcgccggacatcacccccatcatcctggcggcgcactgccagaaatacgaagtggtgcacatgctgctgatgaagggtgccaggatcgagcggccgcacgactatttctgcaagtgcggggactgcatggagaagcagaggcacgactccttcagccactcacgctcgaggatcaatgcctacaaggggctggccagcccggcttacctctcattgtccagcgaggacccggtgcttacggccctagagctcagcaacgagctggccaagctggccaacatagagaaggagttcaagaatgactatcggaagctctccatgcaatgcaaagactttgtagtgggtgtgctggatctctgccgagactcagaagaggtagaagccattctgaatggagatctggaatcagcagagcctctggaggtacacaggcacaaagcttcattaagtcgtgtcaaacttgccattaagtatgaagtcaaaaagtttgtggctcatcccaactgccagcagcagctcttgacgatctggtatgagaacctctcaggcctaagggagcagaccatagctatcaagtgtctcgttgtgctggtcgtggccctgggccttccattcctggccattggctactggatcgcaccttgcagcaggctggggaaaattctgcgaagcccttttatgaagtttgtagcacatgcagcttctttcatcatcttcctgggtctgcttgtgttcaatgcctcagacaggttcgaaggcatcaccacgctgcccaatatcacagttactgactatcccaaacagatcttcagggtgaaaaccacccagtttacatggactgaaatgctaattatggtctgggttcttggaatgatgtggtctgaatgtaaagagctctggctggaaggacctagggaatacattttgcagttgtggaatgtgcttgactttgggatgctgtccatcttcattgctgctttcacagccagattcctagctttccttcaggcaacgaaggcacaacagtatgtggacagttacgtccaagagagtgacctcagtgaagtgacactcccaccagagatacagtatttcacttatgctagagataaatggctcccttctgaccctcagattatatctgaaggcctttatgccatagctgttgtgctcagcttctctcggattgcgtacatcctccctgcaaatgagagctttggccccctgcagatctctcttggaaggactgtaaaggacatattcaagttcatggtcctctttattatggtgttttttgcctttatgattggcatgttcatactttattcttactaccttggggctaaagttaatgctgcttttaccactgtagaagaaagtttcaagactttattttggtcaatatttgggttgtctgaagtgacttccgttgtgctcaaatatgatcacaaattcatagaaaatattggatacgttctttatggaatatacaatgtaactatggtggtcgttttactcaacatgctaattgctatgattaatagctcatatcaagaaattgaggatgacagtgatgtagaatggaagtttgctcgttcaaaactttggttatcctattttgatgatggaaaaacattacctccacctttcagtctagttcctagtccaaaatcatttgtttatttcatcatgcgaattgttaactttcccaaatgcagaaggagaaggcttcagaaggatatagaaatgggaatgggtaactcaaagtccaggttaaacctcttcactcagtctaactcaagagtttttgaatcacacagttttaacagcattctcaatcagccaacacgttatcagcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccattctaattcataaacttagtgagaaactgaatcccagcatgctgagatgtgaatgatgcagcaacctggatttggctttgactatagcacaaatgtgggcaataatatttctaagtatgaaatacttgaaaaactatgatgtaaatttttagtattaactacctttatcatgtgaacctttaaaagttagctcttaatggttttattgttttatcacatgaaaatgcattttatttgtctgctttgacattacagtggcataccattgtgttgaaaagcccaatattactatattattgaaacttttattcattttagagtaaactccacatctttgcactacctgtttgcctccaagagactatcagttccttggggacagggaccatgtcttattcatctttgtgtctccagcatctagtacagtgcctggtatatagtaggtgctcaataaatgttgaaaccaactgaactgccaacaaaataaaaataaaaagtcttcactatgtagcataaaaaaaaaaaaaaaaaaa

SEQ ID NO: 4 - protein, 848 aa, Homo sapiens
MEGSPSLRRMTVMREKGRRQAVRGPAFMFNDRGTSLTAEEERFLDAAEYGNIPVVRKMLEESKTLNVNCVDYMGQNALQLAVGNEHLEVTELLLKKENLARIGDALLLAISKGYVRIVEAILNHPGFAASKRLTLSPCEQELQDDDFYAYDEDGTRFSPDITPIILAAHCQKYEVVHMLLMKGARIERPHDYFCKCGDCMEKQRHDSFSHSRSRINAYKGLASPAYLSLSSEDPVLTALELSNELAKLANIEKEFKNDYRKLSMQCKDFVVGVLDLCRDSEEVEAILNGDLESAEPLEVHRHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVTDYPKQIFRVKTTQFTWTEMLIMVWVLGMMWSECKELWLEGPREYILQLWNVLDFGMLSIFIAAFTARFLAFLQATKAQQYVDSYVQESDLSEVTLPPEIQYFTYARDKWLPSDPQIISEGLYAIAVVLSFSRIAYILPANESFGPLQISLGRTVKDIFKFMVLFIMVFFAFMIGMFILYSYYLGAKVNAAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRIVNFPKCRRRRLQKDIEMGMGNSKSRLNLFTQSNSRVFESHSFNSILNQPTRYQQIMKRLIKRYVLKAQVDKENDEVNEGELKEIKQDISSLRYELLEDKSQATEELAILIHKLSEKLNPSMLRCE